(19) United States
(12) Patent Application Publication
(10) Pub. No.: US 2022/0213298 A1
(43) Pub. Date: Jul. 7, 2022

(54) POROUS CELLULOSE MICROPARTICLES AND METHODS OF MANUFACTURE THEREOF

(71) Applicant: ANOMERA INC., Montreal, Quebec (CA)

(72) Inventors: Mark P. ANDREWS, III, Westmount (CA); Timothy MORSE, Toronto (CA); Monika RAK, Montreal (CA); Zhen HU, Mississauga (CA); Mary BATEMAN, St. Lazare (CA)

(21) Appl. No.: 17/610,121

(22) PCT Filed: May 6, 2020
(86) PCT No.: PCT/CA2020/050605

§ 371 Date: Nov. 9, 2021

Related U.S. Application Data
(60) Provisional application No. 62/846,273, filed on May 10, 2019.

(51) Int. Cl. C08L 1/04; A61K 8/73; A61K 8/02; C08J 9/28; A61Q 19/00

(52) U.S. Cl. CPC C08L 1/04; A61K 8/731; A61K 8/0279; C08J 9/28; A61K 2800/43; C08J 2301/02; A61K 2800/413; A61K 2800/412; A61K 2800/10; A61Q 19/00

(57) ABSTRACT

Porous cellulose microparticles and their use in, inter alias, cosmetic and pharmaceutic preparations are provided. These microparticles comprise cellulose I nanocrystals aggregated together, thus forming the microparticles, and arranged around cavities in the microparticles, thus defining pores in the microparticles. A method of for producing these microparticles is also provided. It involves mixing a suspension of cellulose I nanocrystals with an emulsion of a porogen to produce a mixture comprising a continuous liquid phase in which droplets of the porogen are dispersed and in which the nanocrystals of cellulose I are suspended; spray-drying the mixture to produce microparticles; and if the porogen has not sufficiently evaporated during spray-drying to form pores in the microparticles, evaporating the porogen or leaching the porogen out of the microparticles to form pores in the microparticles.

### CROSS REFERENCE TO RELATED APPLICATIONS

This application claims benefit, under 35 U.S.C. § 119(e), of U.S. provisional application Ser. No. 62/846,273, filed on May 10, 2019. All documents above are incorporated herein in their entirety by reference.

### FIELD OF THE INVENTION

The present invention relates to cellulose microparticles and their methods of use and manufacture. More specifically, the present invention is concerned with porous cellulose microparticles that are made from cellulose nanocrystals by spray-drying.

### BACKGROUND OF THE INVENTION

### Microbeads and Porous Microbeads

Microparticles play important roles in drug delivery, cosmetics and skin care, in fluorescent immunoassay, as micro-carriers in biotechnology, as viscosity modifiers, stationary phases in chromatography, and as abrasives. In these fields, as well as others, microparticles are often referred to as “microbeads”. The cosmetics and personal care industry utilizes microbeads to enhance sensory properties in formulations. Microbeads are used to impart a variety of consumer recognized benefits such as, but not limited to: thickening agent, filler, volumizer, color dispersant, exfoliant, improved product blending, improved skin feel, soft focusing (also known as blurring), product slip, oil uptake, and dry binding. Soft focus or blurring is a property of microbeads due to their ability to scatter light. Oil uptake refers to the capacity of the microbead to absorb sebum form the skin. This property allows cosmetic formulators to design products that impart a mattifying effect to make-up so that a more natural look extends over periods of hours of wear.

Porous microbeads are of interest because they show many unique behaviors not exhibited by dense microbeads. These behaviors include special active molecule (drug) absorption and release kinetics, large specific surface area, and low density. Porous microbeads are differentiated from dense microbeads by the fact that the pores are located not just on the surface, but also in the interior of the microbead. Because of this property, porosity plays an important role in uptake and release kinetics of molecules. Applications of porous microbeads include catalysis, slow release encapsulants for drugs, uptake and binding media, tissue scaffolds, and chromatography. The medical industry uses porous microbeads as tissue engineering scaffolds to proliferate the adhesion and spread of cells. These scaffolds usually carry a drug, like a cell growth factor, to promote proliferation.

Generally speaking, microbeads can be produced from plastics, glass, metal oxides and naturally occurring polymers, like proteins and cellulose. Porous tissue scaffold materials include borate and phosphate glass, silicate and aluminosilicate glass, ceramics, collagen-glucosaminoglycan, calcium phosphate, hydroxyapatite, beta tricalcium phosphate, poly(lactic-co-glycolic acid), carboxymethylcellulose (also known as CMC or cellulose gum). In the cosmetics industry, porous microbeads are conventionally made from plastics, where they are used to impart special effects. Such effects include uptake of oils (sebum, for example) from the skin to impart a mattifying effect.

There is compelling evidence that microbeads made from plastics cause harm to the environment, including damage along the food chain. Increased consumer concern regarding personal health and environmental health has stimulated growth in organic/natural personal care products. Effective organic/natural replacements for traditional products along with societal lifestyle changes are important motivators for widespread adoption not only of “green” personal care products, but also of sustainable ingredients for inks, pigments, coatings, composites and thickeners for paints. Regarding sustainability, it is desirable to use “green chemistry” and “green engineering” methods that use sustainable resources to make microbeads. Use of green methods to produce microbeads is known to reduce the consumption of energy for their manufacture.

Conventionally, porous microparticles are prepared from non-cellulose polymers by the methods of suspension, emulsion and precipitation polymerization. Porous inorganic microparticles can be made by sintering, by phase separation and by spray drying.

### Cellulose and Cellulose Microbeads

Natural cellulose is a hydrophilic semi-crystalline organic polymer. It is a polysaccharide that is produced naturally in the biosphere. It is the structural material of the cell wall of plants, many algae, and fungus-like oomycota. Cellulose is naturally organized into long linear chains of ether-linked poly(β-1,4-glucopyranose) units. These chains assemble by intra- and inter-molecular hydrogen bonds into highly crystalline domains—see FIG. 1. Regions of disordered (amorphous) cellulose exist between these crystalline domains (nanocrystals) in the cellulose nanofibrils. Extensive hydrogen bonding among the cellulose polymer chains makes cellulose extremely resistant to dissolution in water and most organic solvents, and even many types of acids.

Cellulose can exist in several crystalline polymorphs. Among them, cellulose I is the most common as it is the naturally occurring polymorph. Cellulose II is less common, though it is more thermodynamically stable than cellulose I. When manipulating cellulose, for example to make microparticles, the dissolution of cellulose followed by its crystallization forms the thermodynamically stable cellulose II, not the naturally occurring cellulose I. The main differences between celluloses I and II are shown in FIGS. 2A) and B).

Cellulose is widely used as a nontoxic excipient in food and pharmaceutical applications. In medical applications like oral drug delivery, drugs are mixed with cellulose powder (usually microcrystalline cellulose powder) and other fillers and converted by extrusion and spheronisation. Extrusion and spheronisation yield granulate powders. Porous microbeads can be used to make a chromatographic support stationary phase for size exclusion chromatography and as selective adsorbents for biological substances such as proteins, endotoxins, and viruses.

The literature on cellulose microparticles teaches that it may be advantageous to modify cellulose microparticles with chemical compounds to adjust their functionality. These steps are conventionally accomplished by etherification, esterification, oxidation and polymer grafting. Accordingly, it is possible to introduce alkenes, oxiranes, amines, carbonyls, tosyl groups, and other reactive functionalities useful to immobilize proteins. In some cases, polysaccharides derived from starch have been included and subsequently hydrolyzed with amylases. To prevent excessive swelling, disintegration or dissolution, cellulose can be crosslinked after regeneration. Epichlorohydrin is most commonly used for this purpose. The addition of ionic groups may be desired for ion exchange and other purposes. Carboxylate groups offer weak acidity, whereas sulfate and sulfonate groups are comparably stronger. Cationic cellulose microparticles have been prepared by binding tertiary amines. Post-modification of cellulose microparticles in this manner has the disadvantage that the reactions are heterogeneous, sometimes aggressive causing damage to the microparticle, and result in a gradient density of functional groups that decreases towards the interior of the particle.

Conventionally, to make a cellulose microbead, semi-crystalline cellulose is first dissolved, which means that the original crystalline structure of the cellulose (cellulose I) is lost. Dissolution can be achieved (a) by chemical modification, (b) by solvation in aqueous or protic systems, or (c) by dissolution in non-aqueous, non-derivatizing media. An example of (a) is the widely used viscose process that reacts cellulose with strong base (alkali) and carbon disulphide to make an unstable xanthate. The resulting cellulose can then be shaped, for example, into a sphere or another shape. An example of (b) is the reaction of cellulose with a methylammonium cation such as Cuoxen ([Cu(NH₂(CH₂)₂NH₂)₂][OH]₂), or with sodium hydroxide (NaOH) in the process of mercerization. When NaOH/H₂O is used to dissolve cellulose with low crystallinity and degree of polymerization, it may be exploited to shape the natural polymer; dissolution is accompanied by gelation, which can be used to prepare aerogels with geometric shapes like cylinders and spheres. An example of (c) is the reaction of cellulose with an ionic liquid such as 1-ethyl-3-methylimidazolium acetate (EMIMAc). In all of the above, it is necessary to dissolve naturally occurring cellulose in order to make a shaped object. In other cases, native cellulose is dissolved and then converted to a derivative of cellulose in the form of esters like cellulose acetate, cellulose butyrate, cellulose carbamate, cellulose xanthate, and carboxymethyl cellulose, or it is converted to a silylated form called trimethylsilylcellulose. Any of these cellulose derivatives can be used as the starting material to make cellulose microbeads, though not necessarily porous microbeads. The processes (a) to (c) require that cellulose be dissolved and that the dissolved cellulose be converted to microbeads by the processes of dropping, jet cutting, spin drop atomization, spinning disc atomization, spray drying or dispersion.

All of the above processes to make cellulose microbeads, and porous cellulose microbeads, require that cellulose be dissolved to make viscose, or they require other multistep processes involving chemical reactions and input of energy to make cellulose acids, cellulose esters or silylated cellulose. These steps are required to convert natural semi-crystalline cellulose of type I into a solvent-soluble polysaccharide that can be converted to the intended derivative to make microbeads.

In the case of dissolved cellulose, the porosity of produced microparticles is usually controlled by a coagulation process. Beads prepared from higher dissolved cellulose concentrations yield less porous structures. Temperature and composition of the coagulating medium influence morphology, internal surface area, and pore size distribution. “Blowing agents” like NaHCO₃ and azodicarbonamide will decompose in cellulose microparticles and liberate gases to create pores. Overall, it is difficult to make porous cellulose microparticles with porosity that can be controlled at will.

Cellulobeads® D-5 to D-100 are 5 to 100 μm spherical cellulose microbeads manufactured by Daito Kasei. The method of manufacture can be described as follows: semicrystalline solid cellulose from wood pulp is dissolved in strong base to make viscose (viscose process). Calcium carbonate (to inhibit aggregation and control sphere size) is combined with an aqueous basic solution of an anionic polymer like sodium polyacrylate, which is subsequently added to the viscose. This step yields a dispersion of viscose fine particles. These particles are heated to aggregate the viscose, then neutralized with acid and separated by filtration—see US patent publication no. 2005/0255135 A1 and International patent publication no. WO 2017\101103 A1, incorporated herein by reference. The particles produced in that manner are composed of cellulose II, which is not in the form of nanocrystals.

International patent publication no. WO 20161015148 A1, incorporated herein by reference, teaches how to produce nanocrystals of nanocrystalline cellulose and then to aggregate these nanocrystals into roughly spherical microbeads by spray-drying. The cellulose microbeads thus produced have a limited porosity.

### SUMMARY OF THE INVENTION

In accordance with the present invention, there is provided:
1. Porous cellulose microparticles comprising:
   cellulose I nanocrystals aggregated together, thus forming the microparticles, and arranged around cavities in the microparticles, thus defining pores in the microparticles.
2. The microparticles of item 1, wherein the microporous particles have a castor oil uptake of about 60 ml/100 g or more.
3. The microparticles of item 1 or 2, wherein the castor oil uptake is about 65, about 75, about 100, about 125, about 150, about 175, about 200, about 225, or about 250 ml/100 g or more.
4. The microparticles of any one of items 1 to 3, wherein the microporous particles have a surface area of about 30 m²/g or more.
5. The microparticles of any one of items 1 to 4, wherein the surface area is about 45, about 50, about 75, about 100, about 125, or about 150 m²/g or more.
6. The microparticles of any one of items 1 to 5, wherein the microparticles are spheroidal or hemi-spheroidal.
7. The microparticles of any one of items 1 to 6, wherein the microparticles have a sphericity, ψ, of about 0.85 or more, preferably about 0.90 or more, and more preferably about 0.95 or more.
8. The microparticles of any one of items 1 to 7, wherein the microparticles are essentially free from each other.
9. The microparticles of any one of items 1 to 8, wherein the microparticles are in the form of a free-flowing powder.
10. The microparticles of any one of items 1 to 9, wherein the microparticles are from about 1 μm to about 100 μm in diameter, preferably about 1 μm to about 25 μm, more preferably about 2 μm to about 20 μm, and yet more preferably about 4 μm to about 10 μm.
11. The microparticles of any one of items 1 to 10, wherein the microparticles have a size distribution (D₁₀/D₉₀) of about 5/15 to about 5/25.
12. The microparticles of any one of items 1 to 11, wherein the pores are from about 10 nm to about 500 nm in size, preferably from about 50 to about 100 nm in size.
13. The microparticles of any one of items 1 to 12, wherein the cellulose I nanocrystals are from about 50 nm to about 500 nm, preferably from about 80 nm to about 250 nm, more preferably from about 100 nm to about 250 nm, and yet more preferably from about 100 to about 150 nm in length.
14. The microparticles of any one of items 1 to 13, wherein the cellulose I nanocrystals are from about 2 to about 20 nm in width, preferably about 2 to about 10 nm and more preferably from about 5 nm to about 10 nm in width.
15. The microparticles of any one of items 1 to 14, wherein the cellulose I nanocrystals have a crystallinity of at least about 50%, preferably at least about 65% or more, more preferably at least about 70% or more, and most preferably at least about 80%.
16. The microparticles of any one of items 1 to 15, wherein the cellulose I nanocrystals are functionalized cellulose I nanocrystals.
17. The microparticles of any one of items 1 to 16, wherein the cellulose I nanocrystals are sulfated cellulose I nanocrystals and salts thereof, carboxylated cellulose I nanocrystals and salts thereof, cellulose I nanocrystals chemically modified with other functional groups, or a combination thereof.
18. The microparticles of item 17, wherein the salt of sulfated cellulose I nanocrystals and carboxylated cellulose I nanocrystals is the sodium salt thereof.
19. The microparticles of item 17 or 18, wherein the other functional groups are esters, ethers, quaternized alkyl ammonium cations, triazoles and their derivatives, olefins and vinyl compounds, oligomers, polymers, cyclodextrins, amino acids, amines, proteins, or polyelectrolytes.
20. The microparticles of any one of items 1 to 19, wherein the cellulose I nanocrystals in the microparticles are carboxylated cellulose I nanocrystals and salts thereof, preferably carboxylated cellulose I nanocrystals or cellulose I sodium carboxylate salt, and more preferably carboxylated cellulose I nanocrystals.
21. The microparticles of any one of items 1 to 20, comprising one or more further components in addition to cellulose I nanocrystals.
22. The microparticles of item 21, wherein the one or more further components are coated on the cellulose I nanocrystals, deposited on the walls of the pores in the microparticles, or interspersed among the nanocrystals.
23. The microparticles of item 22, wherein at least one of the further components is coated on the cellulose I nanocrystals.
24. The microparticles of item 23, wherein the cellulose I nanocrystals are coated with a polyelectrolyte layer, or a stack of polyelectrolyte layers with alternating charges, preferably one polyelectrolyte layer.
25. The microparticles of item 24, wherein the cellulose I nanocrystals are coated with one or more dyes.
26. The microparticles of item 25, wherein the one or more dyes are located:
   directly on the surface of the cellulose I nanocrystals or
   on top of a polyelectrolyte layer, or a stack of polyelectrolyte layers with alternating charges, preferably one polyelectrolyte layer.
27. The microparticles of item 25 or 26, wherein the one or more dyes comprises a positively charged dye.
28. The microparticles of item 27, wherein the positively charged dye is Red dye #2GL, Light Yellow dye #7GL, or a mixture thereof.
29. The microparticles of any one of items 25 to 28, wherein the one or more dyes comprises a negatively charged dye.
30. The microparticles of item 29, wherein the negatively charged dye is D&C Red dye #28, FD&C Red dye #40, FD&C Blue dye #1 FD&C Blue dye #2, FD&C Yellow dye #5, FD&C Yellow dye #6, FD&C Green dye #3, D&C Orange dye #4, D&C Violet dye #2, phloxine B (D&C Red dye #28), and Sulfur Black #1. Preferred dyes include phloxine B (D&C Red dye #28), FD&C blue dye #1, FD&C yellow dye #5, or a mixture thereof.
31. The microparticles of any one of items 24 to 30, wherein the polyelectrolyte layer is, or the stack of polyelectrolyte layers comprises, a layer of a polyanion.
32. The microparticles of item 31, wherein the polyanion is a copolymer of acrylamide with acrylic acid and copolymers with sulphonate-containing monomers, such as the sodium salt of 2-acrylamido-2-methyl-propane sulphonic acid (AMPS® sold by The Lubrizol® Corporation).
33. The microparticles of any one of items 24 to 33, wherein the polyelectrolyte layer is, or the stack of polyelectrolyte layers comprises, a layer of a polycation.
34. The microparticles of item 33, wherein the polycation is a cationic polysaccharide (such as cationic chitosans and cationic starches), quaternized poly-4-vinylpyridine, poly-2-methyl-5-vinylpyridine, poly(ethyleneimine), poly-L-lysine, a poly(amidoamine), a poly(amino-co-ester), or a polyquaternium.
35. The microparticles of item 34, wherein the polycation is polyquaternium-6, which is poly(diallyldimethylammonium chloride) (PDDA).
36. The microparticles of any one of items 22 to 35, wherein at least one of the further components is deposited on the walls of the pores in the microparticles.
37. The microparticles of item 36, wherein one or more emulsifiers, surfactants, and/or co-surfactants are deposited on the walls of the pores in the microparticles.
38. The microparticles of item 36 or 37, wherein a chitosan, a starch, methylcellulose, gelatin, alginate, albumin, gliadin, pullulan, and/or dextran are deposited on the walls of the pores in the microparticles.
39. The microparticles of any one of items 22 to 38, wherein at least one of the further components is interspersed among the nanocrystals.
40. The microparticles of item 39, wherein a protein, such as silk fibroin or gelatin, preferably fibroin, is interspersed among the nanocrystals.
41. A cosmetic preparation comprising the microparticles of any one of items 1 to 40 and one or more cosmetically acceptable ingredients.
42. The cosmetic preparation of 41 being a product destined to be applied to:
   the face, such as skin-care creams and lotions, cleansers, toners, masks, exfoliants, moisturizers, primers, lipsticks, lip glosses, lip liners, lip plumpers, lip balms, lip stains, lip conditioners, lip primers, lip boosters, lip butters, towelettes, concealers, foundations, face powders, blushes, contour powders or creams, highlight powders or creams, bronzers, mascaras, eye shadows, eye liners, eyebrow pencils, creams, waxes, gels, or powders, or setting sprays;
   the body, such as perfumes and colognes, skin cleansers, moisturizers, deodorants, lotions, powders, baby products, bath oils, bubble baths, bath salts, body lotions, or body butters;
   the hands/nails, such as fingernail and toe nail polish, and hand sanitizer; or
   the hair, such as shampoo and conditioner, permanent chemicals, hair colors, or hairstyling products (e.g. hair sprays and gels).
43. Use of the microparticles of any one of items 1 to 40, or the cosmetic of 41 or 42, to absorb sebum on the skin.
44. Use of the microparticles of any one of items 1 to 40, or the cosmetic of 41 or 42, to provide a soft-focus effect on the skin.
45. Use of the microparticles of any one of items 1 to 40, or the cosmetic of 41 or 42, to provide a haze effect on the skin.
46. Use of the microparticles of any one of items 1 to 40, or the cosmetic of 41 or 42, to provide a mattifying effect on the skin.
47. Use of the microparticles of any one of items 1 to 40 as a support for affinity or immunoaffinity chromatography or for solid phase chemical synthesis.
48. Use of the microparticles of any one of items 1 to 40 in waste treatment.
49. A method for producing the porous cellulose microparticles of any one of items 1 to 40, the method comprising the steps of:
   a) providing a suspension of cellulose I nanocrystals;
   b) providing an emulsion of a porogen,
   c) mixing the suspension with the emulsion to produce a mixture comprising a continuous liquid phase in which droplets of the porogen are dispersed and in which the nanocrystals are suspended;
   d) spray-drying the mixture to produce microparticles; and
   e) if the porogen has not sufficiently evaporated during spray-drying to form pores in the microparticles, evaporating the porogen or leaching the porogen out of the microparticles to form pores in the microparticles.
50. The method of item 49, further comprising the step of establishing a calibration curve of the porosity of microparticles to be produced as a function of the emulsion volume to cellulose I nanocrystals mass ratio of the mixture of step c).
51. The method of item 50, further comprising the step of using the calibration curve to determine the emulsion volume to cellulose I nanocrystals mass ratio of the mixture of step c) allowing to produce microparticles with a desired porosity.
52. The method of any one of items 49 to 51, further comprising the step of adjusting the emulsion volume to cellulose I nanocrystals mass ratio of the mixture of step c) in order to produce microparticles with a desired porosity.
53. The method of item 49, further comprising the step of establishing a calibration curve of the oil uptake of microparticles to be produced as a function of the emulsion volume to cellulose I nanocrystals mass ratio of the mixture of step c).
54. The method of item 53, further comprising the step of using the calibration curve to determine the emulsion volume to cellulose I nanocrystals mass ratio of the mixture of step c) allowing to produce microparticles with a desired oil uptake.
55. The method of any one of items 49, 53, and 54, further comprising the step of adjusting the emulsion volume to cellulose I nanocrystals mass ratio of the mixture of step c) in order to produce microparticles with a desired oil uptake.
56. The method of any one of items 49 to 55, wherein a liquid phase of the suspension in step a) is water or a mixture of water with one or more water-miscible solvent, preferably water, more preferably distilled water.
57. The method of item 56, wherein the water-miscible solvent is acetaldehyde, acetic acid, acetone, acetonitrile, 1,2-, 1,3-, and 1,4-butanediol, 2-butoxyethanol, butyric acid, diethanolamine, diethylenetriamine, dimethylformamide, diemthoxyethane, dimethylsufoxide, ethanol, ethyl amine, ethylene glycol, formic acid, fufuryl alcohol, glycerol, methanol, methanolamine, methyldiethanolamine, N-methyl-2-pyrrolidone, 1-propanol, 1,3- and 1,5-propanediol, 2-propanol, propanoic acid, propylene glycol, pyridine, tetrahydrofuran, triethylene glycol, 1,2-dimethylhydrazine, or a mixture thereof.
58. The method of item 56 or 57, wherein the liquid phase further comprises one or more water-soluble, partially water-soluble, or water-dispersible ingredient.
59. The method of item 58, wherein the water-soluble, partially water-soluble, or water-dispersible ingredient is an acid, a base, a salt, a water-soluble polymer, tetraethoxyorthosilicate (TEOS), or a dendrimer or polymer that make micelles, or a mixture thereof.
60. The method of item 59, wherein the water-soluble polymer is a polymer of the family of divinyl ether-maleic anhydride (DEMA), a poly(vinylpyrrolidine), a pol(vinyl alcohol), a poly(acrylamide), N-(2-hydroxypropyl) methacrylamide (HPMA), poly(ethylene glycol) or one of its derivatives, poly(2-alkyl-2-oxazolines), a dextran, xanthan gum, guar gum, a pectin, a chitosan, a starch, a carrageenan, hydroxypropylmethyl cellulose (HPMC), hydroxypropyl cellulose (HPC), hydroxyethyl cellulose (HEC), sodium carboxy methyl cellulose (Na-CMC), hyaluronic acid (HA), albumin, starch or one of its derivatives, or a mixture thereof.
61. The method of any one of items 49 to 60, wherein the emulsion is an oil-in-water emulsion (O/W), a water-in-oil (W/O) emulsion, a bicontinuous emulsion, or a multiple emulsion; preferably an oil-in-water (O/W) emulsion, a water-in-oil (W/O) emulsion, or an oil-in-water-in-oil (O/W/O) emulsion, and more preferably an oil-in-water (O/W) emulsion.
62. The method of any one of items 49 to 61, wherein the emulsion in step b) is a nanoemulsion.
63. The method of item 62, wherein the nanoemulsion comprises two immiscible liquids, wherein:
   one of the two immiscible liquids is water or an aqueous solution containing one or more salt(s) and/or other water-soluble ingredients, preferably water, and more preferably distilled water, and
   the other of the two immiscible liquids is a water-immiscible organic liquid.
64. The method of item 63, wherein the water-immiscible organic liquid comprises one or more oil, one or more hydrocarbon, one or more fluorinated hydrocarbon, one or more long chain ester, one or more fatty acid, or a mixture thereof.
65. The method of item 64, wherein the one or more oils are an oil of plant origin, a terpene oil, a derivative of these oils, or a mixture thereof.
66. The method of item 65, wherein the oil of plant origin is sweet almond oil, apricot kernel oil, avocado oil, beauty leaf oil, castor oil, coconut oil, coriander oil, corn oil, eucalyptus oil, evening primrose oil, groundnut oil, grapeseed oil, hazelnut oil, linseed oil, olive oil, peanut oil, rye oil, safflower oil, sesame oil, soy bean oil, sunflower oil, wheat germ oil, or a mixture thereof.
67. The method of item 65 or 66, wherein the terpene oil is alpha-pinene, limonene, or a mixture thereof, preferably limonene.
68. The method of any one of items 65 to 67, wherein the one or more hydrocarbon are:
   an alkane, such as heptane, octane, nonane, decane, dodecane, mineral oil, or a mixture thereof, or
   an aromatic hydrocarbon, such as toluene, ethylbenzene, and xylene or a mixture thereof, or a mixture thereof.
69. The method of any one of items 65 to 68, wherein the one or more fluorinated hydrocarbon are perfluorodecalin, perfluorhexane, perfluorooctylbromide, perfluorobutylamine, or a mixture thereof.
70. The method of any one of items 65 to 69, wherein the one or more fatty acid are caprylic, pelargonic, capric, lauric, myristic, palmitic, mergiric, stearic, arachadinic, behenic, palmitolic, oleic, elaidic, raccenic, gadoleic, cetolic, erucic, linoleic, stearidonic, arachidonic, timnodonic, clupanodonic, or cervonic acid, or a mixture thereof.
71. The method of any one of items 65 to 70, wherein the one or more long chain ester is C₁₂-C₁₅ alkyl benzoate, 2-ethylhexyl caprate/caprylate, octyl caprate/caprylate, ethyl laurate, butyl laurate, hexyl laurate, isohexyl laurate, isopropyl laurate, methyl myristate, ethyl myristate, butyl myristate, isobutyl myristate, isopropyl myristate, 2-ethylhexyl monococoate, octyl monococoate, methyl palmitate, ethyl palmitate, isopropyl palmitate, isobutyl palmitate, butyl stearate, isopropyl stearate, isobutyl stearate, isopropyl isostearate, 2-ethylhexyl pelargonate, octyl pelargonate, 2-ethylhexyl hydroxy stearate, octyl hydroxy stearate, decyl oleate, diisopropyl adipate, bis(2-ethylhexyl) adipate, dioctyl adipate, diisocetyl adipate, 2-ethylhexyl succinate, octyl succinate, diisopropyl sebacate, 2-ethylhexyl malate, octyl malate, pentaerythritol caprate/caprylate, 2-ethylhexyl hexanoate, octyl hexanoate, octyldodecyl octanoate, isodecyl neopentanoate, isostearyl neopentanoate, isononyl isononanoate, isotridecyl isononanoate, lauryllactate, myristyllactate, cetyl lactate, myristyl propionate, 2-ethylhexanoate, octyl 2-ethylhexanoate, 2-ethylhexyl octanoate, octyl octanoate, isopropyllauroyl sarcosinate, or a mixture thereof.
72. The method of item 71, wherein the one or more long chain ester is C₁₂-C₁₅ alkyl benzoate, such as that sold by Lotioncrafter® as Lotioncrafter® Ester AB and having CAS no. 68411-27-8, isopropyl myristate, or a mixture thereof.
73. The method of any one of items 63 to 72, wherein the water-immiscible organic liquid is C₁₂-C₁₅ alkyl benzoate, alpha-pinene, or limonene, preferably C₁₂-C₁₅ alkyl benzoate or limonene.
74. The method of any one of items 63 to 73, wherein the water-immiscible organic liquid is present in the nanoemulsion at a concentration in the range of about 0.5 v/v % to about 10 v/v %, preferably about 1 v/v % to about 8 v/v %, the percentages being based on the total volume of the nanoemulsion.
75. The method of any one of items 62 to 74, wherein the nanoemulsion comprises one or more surfactants.
76. The method of item 75, wherein the one or more surfactants are:
   propylene glycol monocaprylate, for example Capryol® 90 sold by Gatte Fossé®,
   lauroyl polyoxyl-32 glycerides and stearoyl polyoxyl-32 glycerides, for example Gelucire® 44/14 and 50/13 sold by Gatte Fossé®,
   glyceryl monostearate, such as that sold by IOI Oleochemical® as Imwitor® 191,
   caprylic/capric glycerides, such as that sold by IOI Oleochemical® as Imwitor® 742,
   isostearyl diglyceryl succinate, such as that sold by IOI Oleochemical® as Imwitor® 780 k,
   glyceryl cocoate, such as that sold by IOI Oleochemical® as Imwitor® 928,
   glycerol monocaprylate, such as that sold by IOI Oleochemical® as Imwitor® 988;
   linoleoyl polyoxyl-6 glycerides, such as that sold as Labrafil® CS M 2125 CS by Gatte Fossé®,
   propylene glycol monolaurate, such as that sold as Lauroglycol® 90 by Gatte Fossé®,
   polyethylene glycol (PEG) with M_{W}>4000;
   polyglyceryl-3 dioleate, such as that sold as Plurol® Oleique CC 947 by Gatte Fossé®,
   polyoxamers (polymers made of a block of polyoxyethylene, followed by a block of polyoxypropylene, followed by a block of polyoxyethylene), such as poloxamer 124 or 128;
   glyceryl ricinoleate, such as that sold by IOI Oleochemical® as Softigen® 701,
   PEG-6 caprylic/capric glycerides, such as that sold by IOI Oleochemical® as Softigen® 767;
   caprylocaproyl polyoxyl-8 glycerides, such as that sold as Labrasol® by Gatte Fossé®,
   polyoxyl hydrogenated castor oils, such as polyoxyl 35 hydrogenated castor oil, such as that sold as Cremophor® EL by Calbiochem, and polyoxyl 60 hydrogenated castor oil; and
   polysorbates, such as polysorbate 20, 60, or 80, like those sold as Tween® 20, 60, and 80 by Croda®, or
   a mixture thereof.
77. The method of item 76, wherein the one or more surfactants is a polysorbate, preferably polysorbate 80.
78. The method of any one of items 75 to 77, wherein the one or more surfactants are present in the nanoemulsion in a surfactants to water-immiscible organic liquid volume ratio of less than 1:1, preferably from about 0.2:1 to about 0.8:1, and more preferably of about 0.75:1.
79. The method of item any one of items 62 to 78, wherein the nanoemulsion comprises one or more co-surfactants.
80. The method of item 79, wherein the one or more co-surfactants are:
   PEG hydrogenated castor oil, for example PEG-40 hydrogenated castor oil such as that sold as Cremophor® RH 40 by BASF® and PEG-25 hydrogenated castor oil such as that sold as Croduret® 25 by Croda®;
   2-(2-ethoxyethoxy)ethanol (i.e. diethylene glycol monoethyl ether), such as Carbitol® sold by Dow® Chemical and Transcutol® P sold by Gatte Fossé®;
   glycerin;
   short to medium-length (C₃ to C₆) alcohols, such as ethanol, propanol, isopropyl alcohol, and n-butanol;
   ethylene glycol;
   poly(ethylene glycol)—for example with an average Mn 25, 300, or 400 (PEG 25, PEG 300, and PEG 400); and
   propylene glycol, or
   a mixture thereof.
81. The method of item 80, wherein the one or more co-surfactants is PEG 25 hydrogenated castor oil.
82. The method of any one of items 79 to 81, wherein the one or more co-surfactants are present in the nanoemulsion in a co-surfactants to surfactants volume ratio in the range about 0.2:1 to about 1:1.
83. The method of any one of items 62 to 82, wherein the nanoemulsion comprises polysorbate 80 as a surfactant and PEG 25 hydrogenated castor oil as a co-surfactant.
84. The method of any one of items 62 to 83, wherein the nanoemulsion is an oil-in-water nanoemulsion.
85. The method of any one of items 62 to 84, wherein the nanoemulsion is:
   an oil-in-water nanoemulsion comprising PEG-25 hydrogenated castor oil, polysorbate 80, C₁₂-C₁₅ alkyl benzoate and water, or
   an oil-in-water nanoemulsion comprising PEG-25 hydrogenated castor oil, polysorbate 80, limonene, and water.
86. The method of any one of items 49 to 61, wherein the emulsion in step b) is a macroemulsion.
87. The method of item 86, wherein the macroemulsion comprises two immiscible liquids, wherein:
   one of the two immiscible liquids is water or an aqueous solution containing one or more salt(s) and/or other water-soluble ingredients, preferably water, and more preferably distilled water, and
   the other of the two immiscible liquids is a water-immiscible organic liquid.
88. The method of item 87, wherein the water-immiscible organic liquid is one or more oil, one or more hydrocarbon, one or more fluorinated hydrocarbon, one or more long chain ester, one or more fatty acid, or a mixture thereof.
89. The method of item 88, wherein the one or more oil is castor oil, corn oil, coconut oil, evening primrose oil, eucalyptus oil, linseed oil, olive oil, peanut oil, sesame oil, a terpene oil, derivatives of these oils, or a mixture there or.

90. The method of item 89, wherein the terpene oil is limonene, pinene, or a mixture thereof. 91. The method of any one of items 88 to 90, wherein the one or more hydrocarbon is:
an alkane, such as heptane, octane, nonane, decane, dodecane, mineral oil, or a mixture thereof, or
an aromatic hydrocarbon, such as toluene, ethylbenzene, xylene, or a mixture thereof, or a mixture thereof.
92. The method of any one of items 88 to 91, wherein the one or more fluorinated hydrocarbons is perfluorodecalin, perfluorhexane, perfluorooctylbromide, perfluorobutylamine, or a mixture thereof.
93. The method of any one of items 88 to 92, wherein the one or more long chain ester is isopropyl myristate.
94. The method of any one of items 88 to 93, wherein the one or more fatty acid is oleic acid.
95. The method of any one of items 87 to 94, wherein the water-immiscible organic liquid is pinene.
96. The method of any one of items 87 to 95, wherein the water-immiscible organic liquid in the macroemulsion is at a concentration in the range of about 0.05 v/v % to about 1 v/v %, preferably about 0.1 v/v % to about 0.8 v/v %, and more preferably about 0.2 v/v %, the percentages being based on the total volume of the macroemulsion.
97. The method of item any one of items 86 to 96, wherein the macroemulsion comprises one or more emulsifiers.
98. The method of item 97, wherein the one or more emulsifiers are:
   methylcellulose,
   gelatin,
   poloxamers (polymers made of a block of polyoxyethylene, followed by a block of polyoxypropylene, followed by a block of polyoxyethylene), such as poloxamer 497;
   mixtures of cetearyl alcohol and coco-glucoside, such as that sold as Montanov® 82 by Seppic®;
   mixtures of palmitoyl proline, magnesium palmitoyl glutamate, and sodium palmitoyl sarcosinate, such as that sold as Sepifeel® One by Seppic®;
   polyoxyl hydrogenated castor oils, such as polyoxyl 35 hydrogenated castor oil, such as that sold as Cremophor® EL by Calbiochem, and polyoxyl 60 hydrogenated castor oil;
   polysorbates, such as polysorbate 20, 60, or 80, like those sold as Tween® 20, 60, and 80 by Croda®, or
   a mixture thereof.
99. The method of item 98, wherein the one or more emulsifiers are methylcellulose, gelatin, a mixture of cetearyl alcohol and coco-glucoside, such as that sold as Montanov® 82, or a mixture of palmitoyl proline, magnesium palmitoyl glutamate, and sodium palmitoyl sarcosinate, such as that sold as Sepifeel® One.
100. The method of any one of items 97 to 99, wherein the one or emulsifiers are present in the macroemulsion at a concentration in the range about 0.05 to about 2 wt %, preferably about 0.1 wt % to about 2 wt %, and more preferably about 0.2 wt % to about 0.5 wt %, the percentages being based on the total weight of the macroemulsion.
101. The method of c any one of items 86 to 100, wherein the macroemulsion comprises one or more co-surfactants.
102. The method of item 101, wherein the one or more co-surfactants are:
   2-(2-ethoxyethoxy)ethanol (i.e. diethylene glycol monoethyl ether), such as Carbitol® sold by Dow® Chemical and Transcutol® P sold by Gatte Fossé®;
   glycerin;
   short to medium-length (C₃ to C₈) alcohols, such as ethanol, propanol, isopropyl alcohol, and n-butanol;
   ethylene glycol;
   poly(ethylene glycol)—for example with an average Mn 250, 300, or 400 (PEG 250, PEG 300, and PEG 400);
   propylene glycol; or
   a mixture thereof.
103. The method of item 102, wherein the one or more co-surfactants are present in the macroemulsion at a concentration in the range of about 0.05 wt % to about 1 wt %, preferably about 0.1 wt % to about 0.8 wt %, and more preferably about 0.2 wt %, the percentages being based on the total weight of the nanoemulsion.
104. The method of any one of items 86 to 103, wherein the macroemulsion is an oil-in-water microemulsion.
105. The method of any one of items 86 to 104, wherein the macroemulsion is:
   an oil-in-water macroemulsion comprising methylcellulose, pinene, and water;
   an oil-in-water macroemulsion comprising gelatin, pinene, and water;
   an oil-in-water macroemulsion comprising a mixture of cetearyl alcohol and coco-glucoside, such as that sold as Montanov® 82, pinene, and water; or
   an oil-in-water macroemulsion comprising a mixture of palmitoyl proline, magnesium palmitoyl glutamate, and sodium palmitoyl sarcosinate, such as that sold as Sepifeel® One, pinene, and water.
106. The method of any one of items 49 to 61, wherein the emulsion in step b) is a microemulsion.
107. The method of item 106, wherein the nanoemulsion comprises two immiscible liquids, wherein:
   one of the two immiscible liquids is water or an aqueous solution containing one or more salt(s) and/or other water-soluble ingredients, preferably water, and more preferably distilled water, and
   the other of the two immiscible liquids is a water-immiscible organic liquid.
108. The method of item 107, wherein the water-immiscible organic liquid is one or more oil, one or more hydrocarbon, one or more fluorinated hydrocarbon, one or more long chain ester, one or more fatty acid, or a mixture thereof.
109. The method of item 108, wherein the one or more oil is castor oil, corn oil, coconut oil, evening primrose oil, eucalyptus oil, linseed oil, olive oil, peanut oil, sesame oil, a terpene oil, a derivative of these oils, or a mixture thereof.
110. The method of item 109, wherein the terpene oil is limonene, pinene, or a mixture thereof.
111. The method of any one of items 108 to 110, wherein the one or more hydrocarbon is:
   an alkane, such as heptane, octane, nonane, decane, dodecane, mineral oil, or a mixture thereof, or
   an aromatic hydrocarbon, such as toluene, ethylbenzene, xylene, or a mixture therefor, or a mixture thereof.
112. The method of any one of items 108 to 111, wherein the one or more fluorinated hydrocarbons is perfluorodecalin, perfluorhexane, perfluorooctylbromide, perfluorobutylamine, or a mixture thereof.
113. The method of any one of items 108 to 112, wherein the one or more long chain ester is isopropyl myristate.
114. The method of any one of items 108 to 113, wherein the one or more fatty acid is oleic acid.
115. The method of any one of items 107 to 114, wherein the water-immiscible organic liquid in the microemulsion is at a concentration in the range of about 0.05 v/v % to about 1 v/v %, preferably about 0.1 v/v % to about 0.8 v/v %, and more preferably about 0.2 v/v %, the percentages being based on the total volume of the microemulsion.
116. The method of any one of items 106 to 115, wherein the microemulsion comprises one or more surfactant.
117. The method of item 116, wherein the one or more surfactant are:
   alkylglucosides of the type CmG1, where Cm represents an alkyl chain consisting of m carbon atoms and G1 represents 1 glucose molecule,
   sucrose alkanoates, such as sucrose monododecanoate,
   polyoxyethylene of the type CmEn, where Cm represents an alkyl chain consisting of m carbon atoms and En represents and ethylene oxide moiety of n units,
   phospholipid derived surfactants, such as lecithin,
   dichain surfactants, like sodium bis(2-ethylhexyl) sulfosuccinate (AOT) and didodecyldimethyl ammonium bromide (DDAB), and
   poloxamers (i.e. polymers made of a block of polyoxyethylene, followed by a block of polyoxypropylene, followed by a block of polyoxyethylene), such as poloxamer 497, or
   a mixture thereof.
118. The method of item 116 or 117, wherein the one or more surfactant are present in the microemulsion at a concentration in the range of about 0.5 wt % to about 8 wt %, preferably about 1 wt % to about 8 wt %, and more preferably about 6.5 wt %, the percentages being based on the total weight of the microemulsion.
119. The method of item any one of items 106 to 118, wherein the microemulsion comprises one or more co-surfactants.
120. The method of item 119, wherein the one or more co-surfactants are:
   2-(2-ethoxyethoxy)ethanol (i.e. diethylene glycol monoethyl ether), such as Carbitol® sold by Dow® Chemical and Transcutol® P sold by Gatte Fossé®,
   glycerin;
   short to medium-length (C₃ to C₈) alcohols, such as ethanol, propanol, isopropyl alcohol, and n-butanol;
   ethylene glycol;
   poly(ethylene glycol)—for example with an average Mn 250, 300, or 400 (PEG 250, PEG 300, and PEG 400);
   propylene glycol; or
   a mixture thereof.
121. The method of item 119 or 120, wherein the one or more co-surfactants are present in the microemulsion at a concentration in the range of about 0.5 v/v % to about 8 wt %, preferably about 1.0 wt % to about 8 wt %, and more preferably about 6.5 wt %, the percentages being based on the total weight of the microemulsion.
122. The method of any one of items 106 to 121, wherein the microemulsion is an oil-in-water microemulsion.
123. The method of any one of items 49 to 122, wherein the emulsion and the suspension are used in an emulsion volume to cellulose I nanocrystals mass ratio from about 1 to about 30 ml/g to form the mixture of step c).
124. The method of any one of items 49 to 123, wherein the porogen has not sufficiently evaporated during spray-drying to form pores in the microparticles, and wherein step e) is carried out.
125. The method of any one of items 49 to 124, wherein step e) is carried out by evaporating the porogen.
126. The method of item 125, wherein the porogen is evaporated by heating, vacuum drying, fluid bed drying, lyophilization, or any combination of these techniques.
127. The method of any one of items 49 to 126, wherein step e) is carried out by leaching the porogen out of the microparticles.
128. The method of item 127, wherein the porogen is leached out of the microparticles by exposing the microparticles to a liquid that is a solvent for the porogen while being a non-solvent for the cellulose I nanocrystals.
129. The method of any one of items 49 to 123, wherein the porogen has sufficiently evaporated during spray-drying to form pores in the microparticles, and wherein step e) is not carried out.

### BRIEF DESCRIPTION OF THE DRAWINGS

In the appended drawings:

FIG. 1 is a schematic representation of cellulose fibers, fibrils, nanofibrils (CNF), and nanocrystals (CNC).

FIG. 2 A) shows the difference between Celluloses I and II in hydrogen bonding patterns.

FIG. 2 B) shows the difference between Celluloses I and II in cellulose chain arrangements.

FIG. 3 is a scanning electron micrograph (SEM) of the microparticles of Example 1.

FIG. 4 is a SEM of the microparticles of Example 2.

FIG. 5 is a SEM of the microparticles of Example 3.

FIG. 6 is a SEM of the microparticles of Comparative Example 1.

FIG. 7 shows in the oil uptake of the microparticles of the Example 1-3 as a function of the ratio of the volume of nanoemulsion (ml) to the total weight of CNC (g).

FIG. 8 shows the mattifying effect of the microparticles of the Example 1-3 and comparative and various conventional products.

FIG. 9 is a SEM of the microparticles of Example 4.

FIG. 10 is a SEM of the microparticles of Example 5.

FIG. 11 is a SEM of the microparticles of Example 6.

FIG. 12 is a SEM of the microparticles of Example 7.

FIG. 13 is a SEM of the microparticles of Example 8.

### DETAILED DESCRIPTION OF THE INVENTION

### Porous Cellulose Microparticles

Turning now to the invention in more details, there is provided porous cellulose microparticles comprising cellulose I nanocrystals aggregated together, thus forming the microparticles, and arranged around cavities in the microparticles, thus defining pores in the microparticles.

The porosity of microparticles can be measured by different methods. One such method is the fluid saturation method as described in the US standard ASTM D281-84. In this method, the oil uptake of a porous microparticle powder is measured. An amount p (in grams) of microparticle powder (between about 0.1 and 5 g) is placed on a glass plate or in a small vial and castor oil (or isononyl isononanoate) is added dropwise. After addition of 4 to 5 drops of oil, the oil is incorporated into the powder with a spatula. Addition of the oil is continued until a conglomerate of the oil and powder has formed. At this point the oil is added one drop at a time and the mixture is then triturated with the spatula. The addition of the oil is stopped when a smooth, firm paste is obtained. The measurement is complete when the paste can be spread on a glass plate without cracking or forming lumps. The volume Vs (expressed in ml) of oil is then noted. The oil uptake corresponds to the ratio Vs/p.

In embodiments, the microporous particles of the invention have a castor oil uptake of about 60 ml/100 g or more. In preferred embodiments, the castor oil uptake is about 65, about 75, about 100, about 125, about 150, about 175, about 200, about 225, or about 250 ml/100 g or more.

The porosity of microparticles can also be measured by the BET (Brunauer-Emmett-Teller) method, which is described in the Journal of the American Chemical Society, Vol. 60, p. 309, 1938, incorporated herein by reference. The BET method conforms to the International Standard ISO 5794/1. The BET method yields a quantity called the surface area (m²/g).

In embodiments, the microporous particles of the invention have a surface area of about 30 m²/g or more. In preferred embodiments, the surface area is about 45, about 50, about 75, about 100, about 125, or about 150 m²/g or more.

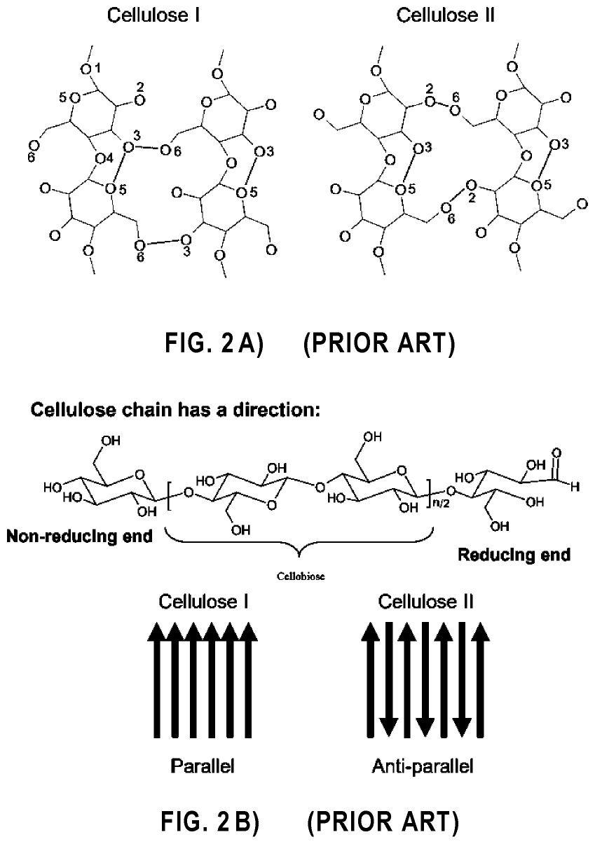

As noted above, the microparticles comprise cellulose I nanocrystals aggregated together. Cellulose I is the naturally occurring polymorph of cellulose. It differs from other polymorphs of cellulose, notably cellulose II as shown in FIG. 2. Cellulose II is the thermodynamically stable cellulose polymorph, cellulose I is not. This means that when cellulose is dissolved, for example during the viscose process, and then crystallized, the resulting cellulose will be cellulose II, not cellulose I. To procure microparticles containing cellulose I, one must start from naturally occurring cellulose and use a manufacturing process that does not break up the crystalline phase in the cellulose; in particular, it must not include dissolution of the cellulose. Such a manufacturing process is provided in the next section.

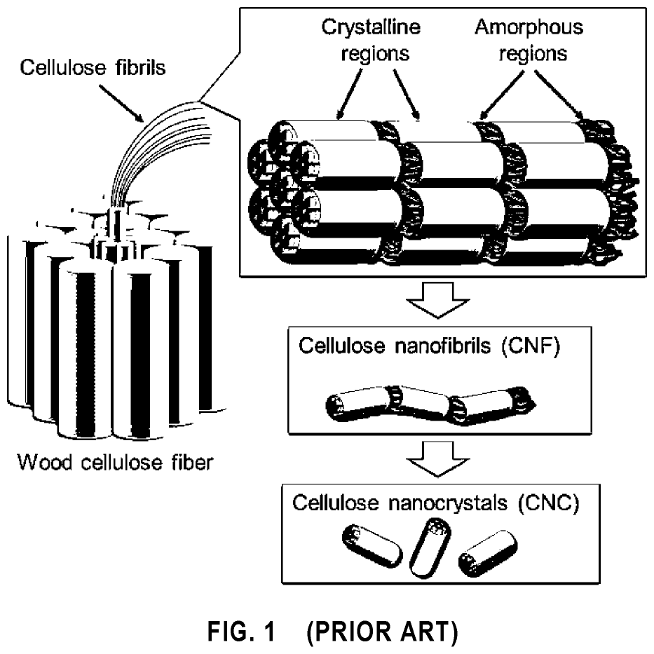

As noted above and shown in FIG. 1, cellulose fibers are made of fibrils. Those fibrils are basically bundles of nanofibrils, each nanofibril containing crystalline cellulose domains separated amorphous cellulose domains. These crystalline cellulose domains can be liberated by removing the amorphous cellulose domains, which yields cellulose nanocrystals—and more specifically of cellulose I nanocrystals if the method employed did not cause the breakup of the cellulose crystalline phase. Cellulose nanocrystals (CNC) are also referred to as crystalline nanocellulose (CNC) and nanocrystalline cellulose (NCC). As shown in FIG. 1, cellulose nanocrystals (CNC) significantly differ from cellulose nanofibrils (CNF).

In embodiments, the microparticles are spheroidal or hemi-spheroidal. Herein, a “spheroid” is the shape obtained by rotating an ellipse about one of its principal axes. Spheroids include spheres (obtained when the ellipse is a circle). Herein, a “hemispheroid” is about one half of a spheroid. The deviation from the shape of a sphere can be determined by an instrument that performs image analysis, such as a Sysmex FPIA-3000. Sphericity is the measure of how closely the shape of an object approaches that of a mathematically perfect sphere. The sphericity, Ψ, of a particle is the ratio of the surface area of a sphere (with the same volume as the particle) to the surface area of the particle. It can be calculated using the following formula:

$$Ψ = \frac{{π}^{1 / 3} - {( 6 {V}_{p} )}^{2 / 3}}{{A}_{p}}$$

wherein Vₚ is the volume of the particle and Aₚ is the surface area of the particle. In embodiments, the sphericity, Ψ, of the microparticles of the invention is about 0.85 or more, preferably about 0.90 or more, and more preferably about 0.95 or more.

In embodiments, the microparticles are typically free from each other, but some of them may be peripherally fused with other microparticles.

In embodiments, the microparticles are in the form of a free-flowing powder.

In embodiments, the microparticles are from about 1 μm to about 100 μm in diameter, preferably about 1 μm to about 25 μm, more preferably about 2 μm to about 20 μm, and yet more preferably about 4 μm to about 10 μm. For cosmetic application, preferred sizes are about 1 μm to about 25 μm, preferably about 2 μm to about 20 μm, and more preferably about 4 μm to about 10 μm.

In embodiments, the microparticles have a size distribution (D₁₀/D₉₀) of about 5/15 to about 5/25, i.e. about 0.33 to about 0.2.

In the microparticles of the invention, the cellulose I nanocrystals are aggregated together (thus forming the microparticles) and are arranged around cavities in the microparticles (thus defining the pores in the microparticles).

As will be explained in the section entitled “Method for Producing the Porous Cellulose Microparticles” below, the microparticles of the invention can be produced by aggregating cellulose I nanocrystals together around droplets of a porogen and then removing the porogen, thus leaving behind voids where porogen droplets used to be, i.e. thus creating pores in the microparticles. This results in nanocrystals aggregated together around cavities (formerly porogen droplets) and forming the microparticles themselves as well as defining (i.e. marking out the boundaries of) the pores in the microparticles.

In embodiments, the pores in the microparticles are from about 10 nm to about 500 nm in size, preferably from about 50 to about 100 nm in size.

### Cellulose I Nanocrystals

In embodiment, the cellulose I nanocrystals are from about 50 nm to about 500 nm, preferably from about 80 nm to about 250 nm, more preferably from about 100 nm to about 250 nm, and yet more preferably from about 100 to about 150 nm in length.

In embodiment, the cellulose I nanocrystals are from about 2 to about 20 nm in width, preferably about 2 to about 10 nm and more preferably from about 5 nm to about 10 nm in width.

In embodiment, the cellulose I nanocrystals have a crystallinity of at least about 50%, preferably at least about 65% or more, more preferably at least about 70% or more, and most preferably at least about 80%.

The cellulose I nanocrystals in the microparticles of the invention may be any cellulose I nanocrystals. In particular, the nanocrystals may be functionalized, which means that their surface has been modified to attached functional groups thereon, or unfunctionalized (as they occur naturally in cellulose). The most common methods of manufacturing cellulose nanocrystals typically cause at least some functionalization of the nanocrystals surface. Hence, in embodiments, the cellulose I nanocrystals are functionalized cellulose I nanocrystals.

In embodiments, the cellulose I nanocrystals in the microparticles of the invention are sulfated cellulose I nanocrystals and salts thereof, carboxylated cellulose I nanocrystals and salts thereof, cellulose I nanocrystals chemically modified with other functional groups, or a combination thereof.

Examples of salts of sulfated cellulose I nanocrystals and carboxylated cellulose I nanocrystals include the sodium salt thereof.

Examples of “other functional groups” as noted above include esters, ethers, quaternized alkyl ammonium cations, triazoles and their derivatives, olefins and vinyl compounds, oligomers, polymers, cyclodextrins, amino acids, amines, proteins, polyelectrolytes, and others. The cellulose I nanocrystals chemically modified with these “other functional groups” are well-known to the skilled person. These “other functional groups” are used to impart one or more desired properties to the cellulose nanocrystals including, for example, fluorescence, compatibility with organic solvents and/or polymers for compounding, bioactivity, catalytic function, stabilization of emulsions, and many other features as known to the skilled person.

Preferably, the cellulose I nanocrystals in the microparticles are carboxylated cellulose I nanocrystals and salts thereof, preferably carboxylated cellulose I nanocrystals or cellulose I sodium carboxylate salt, and more preferably carboxylated cellulose I nanocrystals.

Sulfated cellulose I nanocrystals can be obtained by hydrolysis of cellulose with concentrated sulfuric acid and another acid. This method is well-known and described for example in Habibi et al. 2010, Chemical Reviews, 110, 3479-3500, incorporated herein by reference.

Carboxylated cellulose I nanocrystals can produced by different methods for example, TEMPO- or periodate-mediated oxidation, oxidation with ammonium persulfate, and oxidation with hydrogen peroxide. More specifically, the well-known TEMPO oxidation can be used to oxidize cellulose I nanocrystals. Carboxylated cellulose I nanocrystals can be produced directly from cellulose using aqueous hydrogen peroxide as described in WO 2016/015148 A1, incorporated herein by reference. Other methods of producing carboxylated cellulose I nanocrystals from cellulose include those described in WO 2011/072365 A1 and WO 2013/000074 A1, both incorporated herein by reference.

The cellulose I nanocrystals modified with the “other functional groups” noted above can be produced from sulfated and/or carboxylated CNC (without dissolving the crystalline cellulose) as well-known to the skilled person.

### Optional Components in the Microparticles

In embodiments the microparticles comprise one or more further components in addition to cellulose I nanocrystals. For example, the one or more further components can coated on the cellulose I nanocrystals, deposited on the walls of the pores in the microparticles, interspersed among the nanocrystals.

### Nanocrystal Coating

The cellulose I nanocrystals can be coated before manufacturing the microparticles. As a result, the component(s) of this coating will remain around the nanocrystals, as a coating, in the microparticles. Thus, in embodiments, the nanocrystals in the microparticles are coated.

This is particularly useful to impart a binding effect to the nanocrystals to strengthen the microparticles. Indeed, the very highly porous microparticles may be more brittle, which is generally undesirable and can be counteracted using a binder. In embodiments, this coating is a polyelectrolyte layer, or a stack of polyelectrolyte layers with alternating charges, preferably one polyelectrolyte layer.

Indeed, the surface of the nanocrystals is typically charged. For example, sulfated cellulose I nanocrystals and carboxylated cellulose I nanocrystals and their salts typically have a negatively charged surface. This surface can thus be reacted with one or more polycation (positively charged) that will electrostatically attach itself to, and form a polycation layer on, the surface of the nanocrystals. Conversely, nanocrystals with positively charged surfaces can be coated with a polyanion layer. In both cases, if desired, further polyelectrolyte layers can be similarly formed on top of a previously formed polyelectrolyte layer by reversing the charge of the polyelectrolyte for each layer added.

In embodiments, the polyanions bears groups such as carboxylate and sulfate. Non-limiting examples of such polyanions include copolymers of acrylamide with acrylic acid and copolymers with sulphonate-containing monomers, such as the sodium salt of 2-acrylamido-2-methyl-propane sulphonic acid (AMPS® sold by The Lubrizol® Corporation).

In embodiments, the polycations can bear groups such as quaternary ammonium centers amines. Polycations can be produced in a similar fashion to anionic copolymers by copolymerizing acrylamide with varying proportions of amino derivatives of acrylic acid or methacrylic acid esters. Other examples include cationic polysaccharides (such as cationic chitosans and cationic starches), quaternized poly-4-vinylpyridine and poly-2-methyl-5-vinylpyridine. Non-limiting examples of polycations include poly(ethyleneimine), poly-L-lysine, poly(amidoamine)s and poly(amino-co-ester)s. Other non-limiting examples of polycations are polyquaterniums. “Polyquaternium” is the International Nomenclature for Cosmetic Ingredients (INCI) designation for several polycationic polymers that are used in the personal care industry. INCI has approved different polymers under the polyquaternium designation. These are distinguished by the numerical value that follows the word “polyquaternium”. Polyquaterniums are identified as polyquaternium-1, -2, -4, -5 to -20, -22, -24, -27 to -37, -39, -42, -44 to -47. A preferred polyquaternium is polyquaternium-6, which corresponds to poly(diallyldimethylammonium chloride).

In embodiments, the coating comprises one or more dyes, which would yield a colored microparticles. This dye can be located directly on the nanocrystals surface or on a polyelectrolyte layer.

Non-limiting examples of positively charged dyes include: Red dye #2GL, Light Yellow dye #7GL.

Non-limiting examples of negatively charged dyes include: D&C Red dye #28, FD&C Red dye #40, FD&C Blue dye #1 FD&C Blue dye #2, FD&C Yellow dye #5, FD&C Yellow dye #6, FD&C Green dye #3, D&C Orange dye #4, D&C Violet dye #2, phloxine B (D&C Red dye #28), and Sulfur Black #1. Preferred dyes include phloxine B (D&C Red dye #28), FD&C blue dye #1, and FD&C yellow dye #5.

Substances Interspersed Among the Nanocrystals and/or Deposited on Pore Walls

As explained herein above and below, the microparticles of the invention can be produced by mixing a cellulose I nanocrystal suspension and a porogen emulsion and then using spray-drying to aggregate the nanocrystals together around the porogen droplets and then removing the porogen.

It is well-known (and explained below) that emulsions are typically stabilized using emulsifiers, surfactants, co-surfactants and the like, and that such compounds typically arrange themselves within or at the surface of the porogen droplets. These compounds may or may not be removed during the manufacture of the microparticles. If these compounds are not removed, they will remain in the microparticles along the walls of the pores created by porogen removal. Thus, in embodiments, there are one or more substances deposited on the pore walls in the microparticles. In embodiments, these substances are emulsifiers, surfactants, co-surfactants, such as those described further below. In preferred embodiments, chitosan, a starch, methylcellulose or gelatin is deposited on the pore walls in the microparticles. Other substances include alginate, albumin, gliadin, pullulan, and dextran.

Similarly, both the continuous phase of the porogen emulsion and the liquid phase of nanocrystal suspension can comprise various substances that may not be removed during the manufacture of the microparticles. If these compounds are not removed, they will remain in the microparticles interspersed among the nanocrystals. This is useful to impart a binding effect to the nanocrystals to strengthen the microparticles. Indeed, the very highly porous microparticles may be more brittle, which is generally undesirable and can be counteracted using a binder. In preferred embodiments, a protein, preferably silk fibroin or gelatin, more preferably silk fibroin, is interspersed among the nanocrystals.

### Advantages and Uses of the Microparticles of the Invention

As explained below and as shown in the Example, the porosity of the microparticles can be predictably tuned by adjusting the conditions in which they are manufactured. This, in turns, lead to microparticles with predictably tunable oil uptake, mattifying effect, and refractive index (because these depend on the porosity), which ultimately translate into predictably tunable properties of the microparticles when used, for example in a cosmetic preparation.

The microparticles of the invention are porous (in fact highly or even very highly porous) and thus allows the use of the microparticles to absorb high amounts of a substance. For example, when used in cosmetics, the microparticles with higher oil uptake would be able to absorb more sebum from the skin.

One advantage of the microparticles of the invention is that they are made of cellulose, which is a non-toxic, has desirable mechanical and chemical properties, and is abundant, non-toxic, biocompatible, biodegradable, renewable and sustainable.

### Cosmetic Preparations

The microparticles of the invention can be used in a cosmetic preparation. For example, they can replace plastic microbeads currently used in such preparations. Thus, in another aspect of the invention, there is provided a cosmetic preparation comprising the above microparticles and one or more cosmetically acceptable ingredients.

The nature of these cosmetically acceptable ingredients in the cosmetic preparation is not crucial. Ingredients and formulation well-known to the skilled person may be used to produce the cosmetic preparation.

Herein, a “cosmetic preparation” is a product intended to be rubbed, poured, sprinkled, or sprayed on, introduced into, or otherwise applied to the human body for cleansing, beautifying, promoting attractiveness, or altering appearance. Cosmetics include, but are not limited to, products that can be applied to:
the face, such as skin-care creams and lotions, cleansers, toners, masks, exfoliants, moisturizers, primers, lipsticks, lip glosses, lip liners, lip plumpers, lip balms, lip stains, lip conditioners, lip primers, lip boosters, lip butters, towelettes, concealers, foundations, face powders, blushes, contour powders or creams, highlight powders or creams, bronzers, mascaras, eye shadows, eye liners, eyebrow pencils, creams, waxes, gels, or powders, setting sprays;
the body, such as perfumes and colognes, skin cleansers, moisturizers, deodorants, lotions, powders, baby products, bath oils, bubble baths, bath salts, body lotions, and body butters;
the hands/nails, such as fingernail and toe nail polish, and hand sanitizer; and
the hair, such as shampoo and conditioner, permanent chemicals, hair colors, hairstyling products (e.g. hair sprays and gels).

A cosmetic may be a decorative product (i.e. makeup), a personal care product, or both simultaneously. Indeed, cosmetics are informally divided into:
“makeup” products, which are primarily to products containing color pigments that are intended to alter the user's appearance, and
“personal care” products encompass the remaining products, which are primarily products that support skin/body/hair/hand/nails integrity, enhance their appearance or attractiveness, and/or relieve some conditions that affect these body parts.
   Both types of cosmetics are encompassed within the present invention.

A subset of cosmetics includes cosmetics (mostly personal care products) that are also considered “drugs” because they are intended for use in the diagnosis, cure, mitigation, treatment, or prevention of disease or intended to affect the structure or any function of the body of man or other animals. Examples include antidandruff shampoo, deodorants that are also antiperspirants, products such as moisturizers and makeup marketed with sun-protection claims or anti-acne claims. This subset of cosmetics is also encompassed within the present invention.

Desirable properties and effects can be achieved by a cosmetic preparation comprising the microparticles of the invention. For example, the microparticles confer various optical effects, such as soft-focus effect, haze, and mattifying effect, to the cosmetic preparation. Furthermore, these effects are tunable as explained below.

Optical effects such as soft focus are important benefits conventionally imparted to the skin by spherical particles like silica and plastic microbeads. Moreover, a microparticle that absorbs sebum is desirable because it makes the skin look less shiny and therefore more natural (if the microparticle is non-whitening)—this is referred to as the mattifying effect. Due to environmental concerns, plastic microbeads, including porous plastic microbeads, are banned or are being banned throughout the world, thus there is a need to replace them with porous microparticles that offer the same benefits (tunable oil uptake and mattifying effect), but are friendlier to the environment.

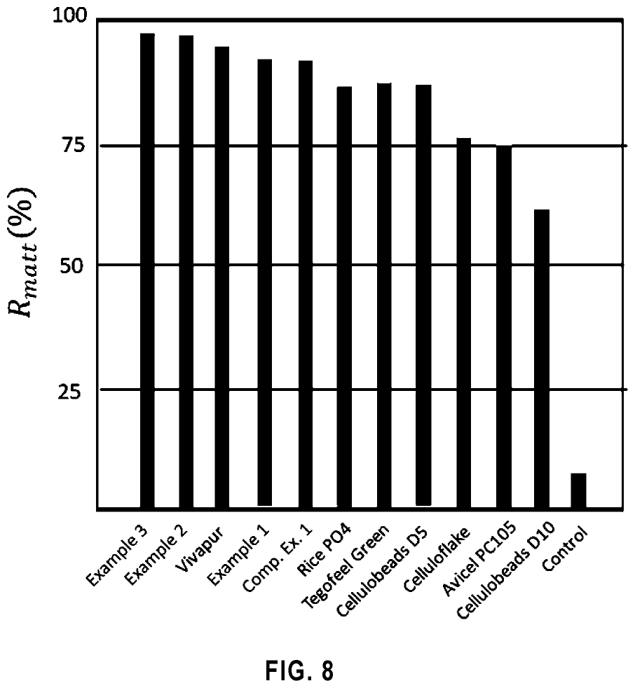

Microparticles with adjustable optical properties, variable oil uptake, or lipophilicity, such as those provided here, are thus advantageous to the cosmetics industry. They can replace plastic microbeads whilst retaining their benefits. Table I (see the Examples below) shows that the refractive index of the microparticles of the invention decreases as the porosity (and hence the oil uptake and the surface area) increases. This change in refractive index affects the appearance of microparticles on the skin. This effect that can be quantitatively described with a parameter called haze. Haze is affected by the refractive index. The microparticles of the invention have an adjustable refractive index so that the benefits of soft focus, haze and other desirable optical features can be predetermined, which makes them a value-added ingredient for cosmetic preparations. Indeed, as shown in Table 1, the refractive index can be predictably tuned by adjusting the manufacturing conditions. Furthermore, as shown in FIG. 8, the microparticles of the invention exhibit a comparable or even better mattifying effect than other cellulose-based materials. This mattifying effect, along with the oil uptake of the microparticles, can be predictably tuned to achieve a specific matte effect—see again Table 1 and FIG. 8. This is very desirable in an ingredient for cosmetic preparations. Because cellulose is hydrophilic, there is a need in the cosmetic industry for cellulose microbeads that are lipophilic. A lipophilic chemical compound will have a tendency to dissolve in, or be compatible with, fats, oils, lipids, and non-polar organic solvents like hexane or toluene. Furthermore, as shown in the examples below, porous cellulose microparticles can be produced that are lipophilic. Lipophilic porous cellulose microparticles also have the advantage that they are more easily formulated in water-in-oil emulsions, and in other largely lipophilic media (like lipsticks).

Moreover, compared with other cellulose ingredients like Avicel® products sold by FMC Biopolymers®, Tego® Feel Green and Tego® Feel C10 sold by Evonik® Industries, or Vivapur® Sensory 5 and Sensory 15S sold by JRS Pharma®, the microparticles of the invention have better feel to the skin. It is believed that this is because these ingredients have irregular shapes and are not made from cellulose nanocrystals, while the microparticles of the invention are more regularly shaped (see above) and are made of cellulose nanocrystals.

### Chromatography Supports

There is a need for porous microparticles for the purification and separation industries. The microparticles of the invention with their adjustable porosity (see the Examples) would be useful for affinity and immunoaffinity chromatography of proteins and for solid phase chemical synthesis, particularly in view of their biocompatibility with enzymes.

### Waste Treatment

The large surface area of the microparticles of the invention (see the Examples) could be useful for metal ion contaminant uptake and the uptake of charged dye molecules known to be carcinogenic (Congo red, for example). It is an advantage that the porous microparticles made according to the invention are charged species, and that the charge can be used to bind oppositely charged ions and that the charge on the microparticle can adjusted from negative (native carboxylate salt or sulfate salt of CNC), to positive (by the adsorption of polyquaternium 6 or chitosan (see the Examples). This obviates the need to impart charge to the microparticle in a post-production process.

It is also an advantage of the present invention that the porosity of the microparticle can be adjusted to create large surface areas for adsorption or porosity to discriminate analytes according to size. Moreover, the large area of the porous microparticles provides an absorbing surface that can be adjusted according to pore size and density.

### Method for Producing the Porous Cellulose Microparticles

In another aspect of the invention, there is provided a method for producing the above porous cellulose microparticles. This method comprises the steps of:
a) providing a suspension of cellulose I nanocrystals;
b) providing an emulsion of a porogen;
c) mixing the suspension with the emulsion to produce a mixture comprising a continuous liquid phase in which droplets of the porogen are dispersed and in which the nanocrystals of cellulose I are suspended;
d) spray-drying the mixture to produce microparticles; and
e) if the porogen has not sufficiently evaporated during spray-drying to form pores in the microparticles, evaporating the porogen or leaching the porogen out of the microparticles to form pores in the microparticles.

During spray-drying, the nanocrystals surprisingly arrange themselves around the porogen droplets. Then, the porogen is removed (creating pores within the microparticles. Porogen removal can happen spontaneously during spray-drying (if the porogen is sufficiently volatile) or otherwise, the porogen is removed in subsequent step e). The use of a volatile porogen has the advantage that there is no need for step e). Surprisingly, during spray-drying, the bigger porogen droplets (those in the micrometer size range) are divided into smaller droplets desirably yielding smaller pores.

One advantage of the above method is that it allows production of microparticles with predictably controlled surface area. The surface area depends on the size of the porogen droplets in the mixture of step c), which can be controlled by adjusting the content and preparation conditions of the emulsion (step b)). Furthermore, and most interestingly, the level of porosity of the microparticles can be controlled by adjusting the total droplet volume to the total nanocrystals weight in the mixture of step c) (i.e. by adjusting the volume of emulsion mixed with the nanocrystal suspension at step c)). To the inventor's knowledge, there are no known methods permitting systematic control over porosity so that cellulose microparticles can be designed to uptake, for example, specific quantities oils. In contrast, as shown in the Examples below, it is possible to establish a calibration curve to predict the porosity/oil uptake of the microparticles according to the present invention based on the above ratio. In other words, this calibration curve permits the production of microparticles with predefined properties.

Thus, in embodiments, the method further comprises the step of establishing a calibration curve of the porosity or oil uptake of microparticles produced as a function of the emulsion volume to cellulose I nanocrystals mass ratio of the mixture of step c). The method of claim may further comprise the step of using the calibration curve to determine the emulsion volume to cellulose I nanocrystals mass ratio of the mixture of step c) allowing to produce microparticles with a desired porosity or oil uptake.

In embodiments, the method further comprises the step of adjusting the emulsion volume to cellulose I nanocrystals mass ratio of the mixture of step c) in order to produce microparticles with a desired porosity or oil uptake.

The method of the invention advantageously produces porous microparticles from cellulose nanocrystals. It does not require that cellulose be dissolved using strong base or other solvents, nor does it require subsequent chemical transformation. The method therefore reduces the number of steps required to make a porous microparticle, requires less energy to do so, and provides a route to porous cellulose microparticles whose production is eco-friendlier. Furthermore, because it does not involve the dissolution of the cellulose or the substantial breakup of its crystalline phase, the method of the invention produces microparticles containing cellulose I (not cellulose II) nanocrystals. In other words, the natural crystalline form of the cellulose is preserved.

Another advantage of the above method is that different types of nanocrystal can be used—carboxylated, sulfated, and chemically modified (see the section of the microparticles themselves for more details). Conventionally, in particular when manufacturing methods that require dissolution of cellulose is used, chemical functional diversity can only be achieved by post-synthesis modification.

Yet another advantage is that a vast range of porogens can be used. (By contrast, porogens cannot be used in the conventional viscose process.) In some cases, when the porogen is sufficiently volatile, there is no need to extract the porogen, which evaporates during spray drying. The porous microparticles are then produced in the gas phase during spray drying.

The method of the invention also allows one to very easily isolate the microparticle produced as a free-flowing powder.

The method advantageously produces microparticles via processes, and from materials, that do not harm the environment.

Step a)—Suspension

Herein, a “suspension” is a mixture that contain solid particles, in the present case the cellulose I nanocrystals, dispersed in a continuous liquid phase. The cellulose I nanocrystals are as defined above.

Typically, such suspensions can be provided by vigorously mixing the nanocrystals with the liquid constituting the liquid phase. Sonication can be used for this mixing as can application of a high-pressure homogenizer or a high speed, high shear rotary mixer.

The liquid phase may be water or a mixture of water with one or more water-miscible solvent, which can for example assist in suspending the nanocrystals in the liquid phase. Non-limiting examples of water-miscible solvents include acetaldehyde, acetic acid, acetone, acetonitrile, 1,2-, 1,3-, and 1,4-butanediol, 2-butoxyethanol, butyric acid, diethanolamine, diethylenetriamine, dimethylformamide, dimethoxyethane, dimethylsufoxide, ethanol, ethyl amine, ethylene glycol, formic acid, fufuryl alcohol, glycerol, methanol, methanolamine, methyldiethanolamine, N-methyl-2-pyrrolidone, 1-propanol, 1,3- and 1,5-propanediol, 2-propanol, propanoic acid, propylene glycol, pyridine, tetrahydrofuran, triethylene glycol, and 1,2-dimethylhydrazine.

The liquid phase may further comprise one or more water-soluble, partially water-soluble, or water-dispersible ingredients. Non-limiting examples of such ingredients include acids, bases, salts, water-soluble polymers, tetraethoxyorthosilicate (TEOS), as well as mixtures thereof. After the microparticles are manufactured by the above method, these ingredients will typically remain within the microparticles interspersed among the nanocrystals.

Non-limiting examples of water-soluble polymers include the family of divinyl ether-maleic anhydride (DEMA), poly(vinylpyrrolidines), pol(vinyl alcohols), poly(acrylamides), N-(2-hydroxypropyl) methacrylamide (HPMA), poly(ethylene glycol) and its derivatives, poly(2-alkyl-2-oxazolines), dextrans, xanthan gum, guar gum, pectins, starches, chitosans, carrageenans, hydroxypropylmethyl cellulose (HPMC), hydroxypropyl cellulose (HPC), hydroxyethyl cellulose (HEC), sodium carboxy methyl cellulose (Na-CMC), hyaluronic acid (HA), albumin, starch and starch-based derivatives. These polymers are useful to impart a binding effect to the nanocrystals to strengthen the microparticles.

Indeed, TEOS may be incorporated into the liquid phase under acid or basic conditions where it can react to make a silica sol particle or react with CNC or combine with CNC and the emulsion to make a cellulose particle that contains silica to improve strength or mechanical stability.

A preferred liquid phase is water, preferably distilled water.

Step b)—Emulsion

Herein, an “emulsion” is a mixture of two or more liquids that are immiscible, in which one liquid, called the dispersed phase, is dispersed in the form of droplets in the other liquid, called the continuous phase. Colloquially, these two liquid phases are referred to, by analogy, as “oil” and “water”.

Types of emulsions include:
oil-in-water emulsions (o/w), in which the dispersed phase is an organic liquid and the continuous phase is water or an aqueous solution,
water-in-oil (w/o) emulsions, in which the dispersed phase is water or an aqueous solution and the continuous phase is an organic liquid,
bicontinuous emulsions, in which the domains of the dispersed phase are interconnected, and
multiple emulsions such as double emulsions including water-in-oil-in-water emulsions (W/O/W) and oil-in-water-in-oil emulsions (O/W/O).

Whether an emulsion turns into any of the above depends on the volume fraction of both phases and the type of surfactant used. The phase volume ratio (ϕ) measures comparative volumes of dispersed and continuous phases. ϕ determines the droplet number and overall stability. Normally, the phase that is present in greater volume becomes the continuous phase. All the above types of the emulsions can be used in the present method. In embodiments, the emulsion in step b) is an oil-in-water (O/W) emulsion, a water-in-oil (W/O) emulsion, or an oil-in-water-in-oil (O/W/O) emulsion. In preferred embodiments, the emulsion in step b) is an oil-in-water (O/W) emulsion.

It will be clear to the skilled person that, in the previous paragraphs, the terms “water” and “oil” used when discussing emulsions are analogies referring to the best-known example of two immiscible liquids. They are not meant to be limitative. “Water” designates in fact an aqueous phase that may contain salt(s) and/or other water-soluble ingredients. Similarly, “oil” refers to any water-immiscible organic liquid. Below, when discussing specific components and preferred components of the emulsions, the terms “oil” and “water” have their regular meaning.

The IUPAC define the following types of emulsions:
nanoemulsions (also called “miniemulsions”) are emulsions in which the droplets of the dispersed phase have diameters in the range from about 50 nm to about 1 μm;
macroemulsions are emulsions in which the droplets of the dispersed phase have a diameter from about 1 to about 100 μm; and
   microemulsions are thermodynamically stable emulsions with dispersed domain diameter varying approximately from about 1 to about 100 nm, usually about 10 to about 50 nm. A microemulsion behaves as a transparent liquid with low viscosity. Its interfaces are disordered. At low oil or water concentration, swollen micelles are present. The swollen micelles are known as microemulsion droplets. At some concentrations, they may form one, two, three or more separate phases that are in equilibrium with each other. These phases may be water-continuous, oil-continuous, or bicontinuous, depending on the concentrations, nature, and arrangements of the molecules present. The structures within these phases may be spheroid (e.g., micelles or reverse micelles), cylinder-like (such as rod-micelles or reverse micelles), plane-like (e.g., lamellar structures), or sponge-like (e.g., bicontinuous). The principal distinction between a microemulsion and a nano- or macroemulsion is neither the size of the droplets nor the degree of cloudiness, but 1) that microemulsions form spontaneously, and 2) that their properties are independent of how they are produced, and 3) that they are thermodynamically stable.

All the above types of the emulsions can be used in the present method. However, macroemulsions that can be used in the present method are limited to those macroemulsions in which the droplets of the dispersed phase have a diameter of at most about 5 μm.

Emulsions are typically stabilized using one or more surfactants, and sometimes co-surfactants and co-solvents, that promote dispersion of the dispersed phase droplets. Microemulsions form spontaneously as a result of ultralow surface tension and a favorable energy of structure formation. Spontaneous formation of the microemulsion is due to the synergistic interaction of surfactant, co-surfactant and co-solvent. Microemulsions are thermodynamically stable. Their particle size does not change over time. Microemulsions can become physically unstable if diluted, acidified or heated. Nanoemulsions and macroemulsions do not form spontaneously. They must be formed by application of shear to a mixture of oil, water and surfactant. Nanoemulsions and macroemulsions are kinetically stable, but thermodynamically unstable: their particle size will increase over time via coalescence, flocculation and/or Ostwald ripening.

Step b) of providing an emulsion of a porogen includes mixing two liquids that are immiscible with each other, optionally together with emulsifiers, surfactant(s), and/or co-surfactant(s) as needed to form an emulsion in which droplets of one of the two immiscible liquids will be dispersed in a continuous phase of the other of the two immiscible liquids.

Herein, the term “porogen” refers to those components of the dispersed phase (one of the immiscible liquids, the emulsifiers, surfactant(s), and/or co-surfactant(s), as well as any other optional additives) that are present in the droplets at steps b) and/or c) and that are removed from the microparticles at steps d) and/or e) thus forming pores in the microparticles. Typically, the porogen includes the liquid (among the two immiscible liquids contained in the emulsion) that forms the droplets. The porogen may also include emulsifiers, surfactant(s), and/or co-surfactant(s); although some of those may also be left behind (i.e. not be a porogen) as explained in the section entitled “Pore Walls” above.

### Nanoemulsions

In embodiments, the emulsion in step b) is a nanoemulsion.

In embodiments, one of the two immiscible liquids forming the nanoemulsion is water or an aqueous solution containing one or more salt(s) and/or other water-soluble ingredients, preferably water, and more preferably distilled water.

In embodiments, the other of the two immiscible liquids is any water-immiscible organic liquid, for example one or more oil, one or more hydrocarbon (either saturated or unsaturated, e.g. olefins), one or more fluorinated hydrocarbons, one or more long chain ester, one or more fatty acid, as well as mixtures thereof.
Non-limiting examples of oils of plant origin include sweet almond oil, apricot kernel oil, avocado oil, beauty leaf oil, castor oil, coconut oil, coriander oil, corn oil, eucalyptus oil, evening primrose oil, groundnut oil, grapeseed oil, hazelnut oil, linseed oil, olive oil, peanut oil, rye oil, safflower oil, sesame oil, soy bean oil, sunflower oil, terpene oils such as alpha-pinene (alpha-2,6,6-trimethylbicyclo[3.1.1]hept-2-ene) and limonene (1-methyl-4-(prop-1-en-2-yl)cyclohex-1-ene), wheat germ oil, and derivatives of these oils.
Non-limiting examples of hydrocarbons include:
   alkanes, such as heptane, octane, nonane, decane, dodecane, and mineral oil and
   aromatic hydrocarbons, such as toluene, ethylbenzene, and xylene.
Non-limiting examples of fluorinated hydrocarbons include perfluorodecalin, perfluorhexane, perfluorooctylbromide, and perfluorobutylamine.
Non-limiting examples of fatty acids include caprylic, pelargonic, capric, lauric, myristic, palmitic, mergiric, stearic, arachadinic, behenic, palmitolic, oleic, elaidic, raccenic, gadoleic, cetolic, erucic, linoleic, stearidonic, arachidonic, timnodonic, clupanodonic, and cervonic acids.
Non-limiting examples of long chain esters include compounds of formula R—C(O)—O—R¹, wherein R and R¹ are saturated or unsaturated hydrocarbons and at least one of R and R¹ contains more than 8 carbon atoms. Specific examples of long chain esters include C₁₂-C₁₅ alkyl benzoate, 2-ethylhexyl caprate/caprylate, octyl caprate/caprylate, ethyl laurate, butyl laurate, hexyl laurate, isohexyl laurate, isopropyl laurate, methyl myristate, ethyl myristate, butyl myristate, isobutyl myristate, isopropyl myristate, 2-ethylhexyl monococoate, octyl monococoate, methyl palmitate, ethyl palmitate, isopropyl palmitate, isobutyl palmitate, butyl stearate, isopropyl stearate, isobutyl stearate, isopropyl isostearate, 2-ethylhexyl pelargonate, octyl pelargonate, 2-ethylhexyl hydroxy stearate, octyl hydroxy stearate, decyl oleate, diisopropyl adipate, bis(2-ethylhexyl) adipate, dioctyl adipate, diisocetyl adipate, 2-ethylhexyl succinate, octyl succinate, diisopropyl sebacate, 2-ethylhexyl malate, octyl malate, pentaerythritol caprate/caprylate, 2-ethylhexyl hexanoate, octyl hexanoate, octyldodecyl octanoate, isodecyl neopentanoate, isostearyl neopentanoate, isononyl isononanoate, isotridecyl isononanoate, lauryllactate, myristyllactate, cetyl lactate, myristyl propionate, 2-ethylhexanoate, octyl 2-ethylhexanoate, 2-ethylhexyl octanoate, octyl octanoate, and isopropyllauroyl sarcosinate. Preferred long chain esters include C₁₂-C₁₅ alkyl benzoate, such as that sold by Lotioncrafter® as Lotioncrafter® Ester AB and having CAS no. 68411-27-8, and isopropyl myristate.
   Preferred water-immiscible organic liquids are C₁₂-C₁₅ alkyl benzoate, alpha-pinene, and limonene (preferably (R)-(+)-limonene), and preferably C₁₂-C₁₅ alkyl benzoate and limonene.

In embodiments, the water-immiscible organic liquid in the nanoemulsion is at a concentration in the range of about 0.5 v/v % to about 10 v/v %, preferably about 1 v/v % to about 8 v/v %, the percentages being based on the total volume of the nanoemulsion.

The nanoemulsion typically comprises one or more surfactants. Non-limiting examples of surfactants include:
propylene glycol monocaprylate, for example Capryol® 90 sold by Gatte Fosse®,
lauroyl polyoxyl-32 glycerides and stearoyl polyoxyl-32 glycerides, for example Gelucire® 44/14 and 50/13 sold by Gatte Fosse®,
glyceryl monostearate, such as that sold by IOI Oleochemical® as Imwitor® 191,
caprylic/capric glycerides, such as that sold by IOI Oleochemical® as Imwitor® 742,
isostearyl diglyceryl succinate, such as that sold by IOI Oleochemical® as Imwitor® 780 k,
glyceryl cocoate, such as that sold by IOI Oleochemical® as Imwitor® 928,
glycerol monocaprylate, such as that sold by IOI Oleochemical® as Imwitor® 988;
linoleoyl polyoxyl-6 glycerides, such as that sold as Labrafil® CS M 2125 CS by Gatte Fosse®,
propylene glycol monolaurate, such as that sold as Lauroglycol® 90 by Gatte Fosse®,
polyethylene glycol (PEG) with M_{W}>4000;
polyglyceryl-3 dioleate, such as that sold as Plurol® Oleique CC 947 by Gatte Fosse®,
polyoxamers (polymers made of a block of polyoxyethylene, followed by a block of polyoxypropylene, followed by a block of polyoxyethylene), such as poloxamer 124 or 128;
glyceryl ricinoleate, such as that sold by IOI Oleochemical® as Softigen® 701,
PEG-6 caprylic/capric glycerides, such as that sold by IOI Oleochemical® as Softigen® 767;
caprylocaproyl polyoxyl-8 glycerides, such as that sold as Labrasol® by Gatte Fosse®,
polyoxyl hydrogenated castor oils, such as polyoxyl 35 hydrogenated castor oil, such as that sold as Cremophor® EL by Calbiochem, and polyoxyl 60 hydrogenated castor oil; and
polysorbates, such as polysorbate 20, 60, or 80, like those sold as Tween® 20, 60, and 80 by Croda®, as well as mixtures thereof. Preferred surfactants include polysorbates. A preferred surfactant is polysorbate 80.

In embodiments, the volume ratio of the surfactant to water-immiscible organic liquid in the nanoemulsion is less than 1:1, preferably about 0.2:1 to about 0.8:1, and more preferably about 0.75:1.

The nanoemulsion may also comprise one or more co-surfactant. Non-limiting examples of co-surfactants include:
PEG hydrogenated castor oil, for example PEG-40 hydrogenated castor oil such as that sold as Cremophor® RH 40 by BASF® and PEG-25 hydrogenated castor oil such as that sold as Croduret® 25 by Croda®;
2-(2-ethoxyethoxy)ethanol (i.e. diethylene glycol monoethyl ether), such as Carbitol® sold by Dow® Chemical and Transcutol® P sold by Gatte Fosse®);
glycerin;
short to medium-length (C₃ to CO alcohols, such as ethanol, propanol, isopropyl alcohol, and n-butanol;
ethylene glycol;
poly(ethylene glycol)—for example with an average Mn 25, 300, or 400 (PEG 25, PEG 300, and PEG 400); and
propylene glycol.
   A preferred co-surfactant is PEG 25 hydrogenated castor oil.

A preferred surfactant/co-surfactant system is polysorbate 80 with PEG 25 hydrogenated castor oil.

In embodiments, the co-surfactant(s) in the nanoemulsion is provided in a volume ratio to surfactant(s) in the range about 0.2:1 to about 1:1.

In preferred embodiments, the water or aqueous solution containing one or more salt(s) and/or other water-soluble ingredients is the continuous phase in the nanoemulsion and the water-immiscible organic liquid is the dispersed phase. In other words, the nanoemulsion is an oil-in-water nanoemulsion.

In preferred embodiments, the nanoemulsion is:
an oil-in-water nanoemulsion comprising PEG-25 hydrogenated castor oil, polysorbate 80, C₁₂-C₁₅ alkyl benzoate and water, or
an oil-in-water nanoemulsion comprising PEG-25 hydrogenated castor oil, polysorbate 80, (R)-(+)-limonene, and water.

Methods of preparing nanoemulsions are well-known to the skilled person. Nanoemulsions can be prepared either by low energy methods or by high energy methods. Low energy methods typically provide smaller and more uniform droplets. High energy methods provide greater control over droplet size and choice of droplet composition, which in turn control stability, rheology and emulsion color. Examples of low energy methods are the phase inversion temperature (PIT) method, the solvent displacement method and the self-nanoemulsion method (i.e. the phase immersion composition (PIC) method). These methods are important because they use the stored energy of the emulsion system to make droplets. For example, a water-in-oil emulsion is usually prepared and then transformed into an oil-in-water nanoemulsion by changing either composition or temperature. The water-in-oil emulsion is diluted dropwise with water to an inversion point or gradually cooled to a phase inversion temperature. The emulsion inversion point and phase inversion temperature cause a significant decrease in the interfacial tension between two liquids, thereby generating very tiny oil droplets dispersed in the water. High energy methods make use of very high kinetic energy by converting mechanical energy to create disruptive forces to break up the oil and water into nanosized droplets. This can be achieved with high shear stirring, ultrasonicators, microfluidizers, and high-pressure homogenizers.

The physical properties of nanoemulsions are commonly assessed by morphology (transmission and scanning electron microscopy), size polydispersity and charge (by dynamic light scattering and zeta potential measurement), and by viscosity. For pharmaceutical applications, skin permeation and bioavailability and pharmacodynamic studies are added.

### Macroemulsions

In embodiments, the emulsion in step b) is a macroemulsion.

In embodiments, one of the two immiscible liquids forming the macroemulsion is water or an aqueous solution containing one or more salt(s) and/or other water-soluble ingredients, preferably water, and more preferably distilled water.

In embodiments, the other of the two immiscible liquids is any water-immiscible organic liquid, for example one or more oil, one or more hydrocarbon (either saturated or unsaturated, e.g. olefins), one or more fluorinated hydrocarbon, one or more long chain ester, one or more fatty acid, etc. as well as mixtures thereof.
Non-limiting examples of oils include castor oil, corn oil, coconut oil, evening primrose oil, eucalyptus oil, linseed oil, olive oil, peanut oil, sesame oil, a terpene oil such as limonene (1-methyl-4-(prop-1-en-2-yl)cyclohex-1-ene) and pinene (2,6,6-trimethylbicyclo[3.1.1]hept-2-ene), and derivatives of these oils.
Non-limiting examples of hydrocarbons include:
   alkanes, such as heptane, octane, nonane, decane, dodecane, and mineral oil and
   aromatic hydrocarbons, such as toluene, ethylbenzene, and xylene.
Non-limiting examples of fluorinated hydrocarbons include perfluorodecalin, perfluorhexane, perfluorooctylbromide, and perfluorobutylamine.
Non-limiting examples of long chain esters include compounds of formula R—C(O)—O—R¹, wherein R and R¹ are saturated or unsaturated hydrocarbons and at least one of R and R¹ contains more than 8 carbon atoms. A preferred long chain ester is isopropyl myristate.
Non-limiting examples of fatty acids include compounds of formula R—COOH, wherein R is long chain hydrocarbon (e.g. containing more than 10 carbon atoms), for example oleic acid.
   A preferred water-immiscible organic liquid is pinene.

In embodiments, the water-immiscible organic liquid in the macroemulsion is at a concentration in the range of about 0.05 v/v % to about 1 v/v %, preferably about 0.1 v/v % to about 0.8 v/v %, and more preferably about 0.2 v/v %, the percentages being based on the total volume of the macroemulsion.

Macroemulsions typically comprise one or more emulsifiers (such as but not limited to surfactants) and optionally one or more co-surfactant.

An “emulsifier” (also known as an “emulgent”) is a substance that stabilizes an emulsion by increasing its kinetic stability. One class of emulsifiers is “surface active agents” (also called “surfactants”). A surfactant is a compound that lowers the interfacial tension between two liquids (i.e. between the dispersed phase and the continuous phase). As such, surfactants form a specific class of emulsifiers.

The macroemulsion thus typically comprises one or more emulsifiers. Non-limiting examples of emulsifiers include:
methylcellulose,
gelatin,
poloxamers (polymers made of a block of polyoxyethylene, followed by a block of polyoxypropylene, followed by a block of polyoxyethylene), such as poloxamer 497;
mixtures of cetearyl alcohol and coco-glucoside, such as that sold as Montanov® 82 by Seppic®;
mixtures of palmitoyl proline, magnesium palmitoyl glutamate, and sodium palmitoyl sarcosinate, such as that sold as Sepifeel® One by Seppic®;
polyoxyl hydrogenated castor oils, such as polyoxyl 35 hydrogenated castor oil, such as that sold as Cremophor® EL by Calbiochem, and polyoxyl 60 hydrogenated castor oil; and
polysorbates, such as polysorbate 20, 60, or 80, like those sold as Tween® 20, 60, and 80 by Croda®.

Preferred emulsifiers include methylcellulose, gelatin, mixtures of cetearyl alcohol and coco-glucoside, such as that sold as Montanov® 82, and mixtures of palmitoyl proline, magnesium palmitoyl glutamate, and sodium palmitoyl sarcosinate, such as that sold as Sepifeel® One.

In embodiments, the emulsifier in the macroemulsion is at a concentration in the range of about 0.05 to about 2 wt %, preferably about 0.1 wt % to about 2 wt %, and more preferably about 0.2 wt % to about 0.5 wt %, the percentages being based on the total weight of the microemulsion.

The macroemulsion may also comprise one or more co-surfactant. Non-limiting examples of co-surfactants include:
2-(2-ethoxyethoxy)ethanol (i.e. diethylene glycol monoethyl ether), such as Carbitol® sold by Dow® Chemical and Transcutol® P sold by Gatte Fossé®,
glycerin;
short to medium-length (C₃ to C₆) alcohols, such as ethanol, propanol, isopropyl alcohol, and n-butanol;
ethylene glycol;
poly(ethylene glycol)—for example with an average Mn 250, 300, or 400 (PEG 250, PEG 300, and PEG 400); and
propylene glycol.

In embodiments, the co-surfactant in the macroemulsion is at a concentration in the range of about 0.05 to about 1 wt %, preferably about 0.1 wt % to about 0.8 wt %, and more preferably about 0.2 wt %, the percentages being based on the total weight of the macroemulsion.

In preferred embodiments, the water or aqueous solution containing one or more salt(s) and/or other water-soluble ingredients is the continuous phase in the macroemulsion and the water-immiscible organic liquid is the dispersed phase. In other words, the macroemulsion is an oil-in-water macroemulsion.

In preferred embodiments, the macroemulsion is:
an oil-in-water macroemulsion comprising methylcellulose, pinene, and water;
an oil-in-water macroemulsion comprising gelatin, pinene, and water;
an oil-in-water macroemulsion comprising a mixture of cetearyl alcohol and coco-glucoside, such as that sold as Montanov® 82, pinene, and water; or
an oil-in-water macroemulsion comprising a mixture of palmitoyl proline, magnesium palmitoyl glutamate, and sodium palmitoyl sarcosinate, such as that sold as Sepifeel® One, pinene, and water.

The preparation of macroemulsions is well-known to the skilled person. Macroemulsions are generally prepared using the low energy methods or the high energy methods described above with regard to nanoemulsions.

### Microemulsions

In embodiments, the emulsion in step b) is a microemulsion.

In embodiments, one of the two immiscible liquids forming the microemulsion is water or an aqueous solution containing one or more salt(s) and/or other water-soluble ingredients, preferably water, and more preferably distilled water.

In embodiments, the other of the two immiscible liquids is any water-immiscible organic liquid, for example one or more oil, one or more hydrocarbon (either saturated or unsaturated, e.g. olefins), one or more fluorinated hydrocarbon, one or more long chain ester, one or more fatty acid, etc. as well as mixtures thereof.
Non-limiting examples of oils include castor oil, corn oil, coconut oil, evening primrose oil, eucalyptus oil, linseed oil, olive oil, peanut oil, sesame oil, a terpene oil such as limonene (1-methyl-4-(prop-1-en-2-yl)cyclohex-1-ene) and pinene (2,6,6-trimethylbicyclo[3.1.1]hept-2-ene), and derivatives of these oils.
Non-limiting examples of hydrocarbons include:
   alkanes, such as heptane, octane, nonane, decane, dodecane, and mineral oil, and
   aromatic hydrocarbons, such as toluene, ethylbenzene, and xylene.
Non-limiting examples of fluorinated hydrocarbons include perfluorodecalin, perfluorhexane, perfluorooctylbromide, and perfluorobutylamine.
Non-limiting examples of long chain esters include compounds of formula R—C(O)—O—R¹, wherein R and R¹ are saturated or unsaturated hydrocarbons and at least one of R and R¹ contains more than 8 carbon atoms. A preferred long chain ester is isopropyl myristate.
Non-limiting examples of fatty acids include compounds of formula R—COOH, wherein R is long chain hydrocarbon (e.g. containing more than 10 carbon atoms), for example oleic acid.

In embodiments, the water-immiscible organic liquid in the microemulsion is at a concentration in the range of about 0.05 v/v % to about 1 v/v %, preferably about 0.1 v/v % to about 0.8 v/v %, and more preferably about 0.2 v/v %, the percentages being based on the total volume of the microemulsion.

Microemulsions typically include surfactants and optionally one or more co-surfactant.

The microemulsion thus typically comprises one or more surfactants. Non-limiting examples of surfactants include:
alkylglucosides of the type CmG1, where Cm represents an alkyl chain consisting of m carbon atoms and G1 represents 1 glucose molecule,
sucrose alkanoates, such as sucrose monododecanoate,
polyoxyethylene of the type CmEn, where Cm represents an alkyl chain consisting of m carbon atoms and En represents and ethylene oxide moiety of n units,
phospholipid derived surfactants, such as lecithin,
dichain surfactants, like sodium bis(2-ethylhexyl) sulfosuccinate (AOT) and didodecyldimethyl ammonium bromide (DDAB), and
poloxamers (i.e. polymers made of a block of polyoxyethylene, followed by a block of polyoxypropylene, followed by a block of polyoxyethylene), such as poloxamer 497.

The required surfactant concentration in a microemulsion is typically several times higher than that in a nanoemulsion or macroemulsion, and typically significantly exceeds the concentration of the dispersed phase. In embodiments, the surfactant in the microemulsion is at a concentration in the range of about 0.5 wt % to about 8 wt %, preferably about 1 wt % to about 8 wt %, and more preferably about 6.5 wt %, the percentages being based on the total weight of the microemulsion.

The microemulsion may also comprise one or more co-surfactant. Non-limiting examples of co-surfactants include:
2-(2-ethoxyethoxy)ethanol (i.e. diethylene glycol monoethyl ether), such as Carbitol® sold by Dow® Chemical and Transcutol® P sold by Gatte Fossé®,
short to medium-length (C₃ to C₈) alcohols, such as ethanol, propanol, isopropyl alcohol, and n-butanol;
ethylene glycol;
poly(ethylene glycol)—for example with an average Mn 250, 300, or 400 (PEG 250, PEG 300, and PEG 400); and
propylene glycol.

In embodiments, the co-surfactant in the microemulsion is at a concentration in the range of about 0.5 v/v % to about 8 wt %, preferably about 1.0 wt % to about 8 wt %, and more preferably about 6.5 wt %, the percentages being based on the total weight of the microemulsion.

In preferred embodiments, the water or aqueous solution containing one or more salt(s) and/or other water-soluble ingredients is the continuous phase in the microemulsion and the water-immiscible organic liquid is the dispersed phase. In other words, the microemulsion is an oil-in-water microemulsion.

The preparation of microemulsion is well-known to the skilled person. Microemulsions typically form spontaneously upon simple mixing of their components due to the synergistic interaction of surfactants, co-surfactants and co-solvents.

Step c)—Mixing

Step c) is the mixing of the suspension with the emulsion to produce a mixture comprising a continuous liquid phase in which droplets of the porogen are dispersed and in which cellulose I nanocrystals are suspended. In other words, the mixture produced is both a porogen emulsion and a nanocrystal suspension.

The continuous liquid phase of the mixture of step c) is provided by the liquid phases of the emulsion and the suspension. Therefore, it is preferred, but not necessary, that these liquid phases be the same, for example water, preferably distilled water.

The dispersed droplets of the porogen in the mixture of step c) are provided by the emulsion of step b).

The suspended cellulose I nanocrystals in the mixture of step c) are provided by the suspension of step a).

As noted above, the level of porosity of the microparticles can be controlled by adjusting the total droplet volume to the total nanocrystals weight in the mixture of step c), i.e. by adjusting the volume of emulsion mixed with the nanocrystal suspension at step c). Generally speaking, the emulsion may be added to the suspension in a volume of emulsion to weight ratio of CNC from about 1 to about 30 ml/g.

Optionally, one or more further components can be added to the mixture at step c). For example, a protein, such as silk fibroin or gelatin, preferably silk fibroin can be added.

The mixture is then stirred with a suitable mixer, such as a VMI mixer.

Step d)—Spray-Drying and Optional Step e)

During step d), the mixture is spray-dried. Generally speaking, spray-drying is a well-known and commonly used method for separating solids content from a liquid medium. Spray-drying separates solutes or suspended matter as solids and the liquid medium into a vapor. The liquid input stream is sprayed through a nozzle into a hot vapor stream and vaporized. Solids form as the vapor quickly leaves the droplets.

In step d), the spray-drying surprisingly causes the cellulose I nanocrystals to arrange themselves around and thus trap the porogen droplets, and to aggregate together into microparticles. Furthermore, if the porogen has a sufficiently low boiling point, spray-drying will then cause the evaporation of the porogen droplets creating pores in the microparticles. If the porogen does not have a sufficiently low boiling point, it will only partially evaporate or not evaporate at all during spray-drying step d). In such cases, to form the desired pores, the porogen will be removed from the microparticles during step e). Hence, step e) is optional. It need only be carried out when the porogen has not (or not sufficiently) evaporated during spray-drying.

Examples of porogens that typically evaporate during spray-drying, i.e. “self-extracting porogens”, include:
terpene oils, such as limonene and pinene, camphene, 3-carene, linalool, caryophyllene, nerolidol, and phytol;
alkanes, such as heptane, octane, nonane, decane, and dodecane;
aromatic hydrocarbons, such as toluene, ethylbenzene, and xylene; and
fluorinated hydrocarbons, such as perfluorodecalin, perfluorhexane, perfluorooctylbromide, and perfluorobutylamine.

Step e) is the evaporation of the porogen or leaching of the porogen out of the microparticles. This can be achieved by any method as long as the integrity of the microparticles is maintained. For example, evaporation can be achieved by heating, vacuum drying, fluid bed drying, lyophilization, or any combination of these techniques. Leaching can be achieved by exposing the microparticles to a liquid that will dissolve the porogen (i.e. it is a porogen solvent) while being a non-solvent for the cellulose I nanocrystals.

### Steps a), b), and c) Carried Out Simultaneously

In embodiments, steps a), b), and c) can be carried simultaneously.

In such embodiments, the mixture of step c) is prepared as a Pickering emulsion, which is both an emulsion and a suspension. Indeed, a Pickering emulsion is an emulsion that is stabilized by solid particles, in the present case, cellulose I nanocrystals, which adsorb onto the interface between the two phases (i.e. around the porogen droplets). In other words, the cellulose nanocrystals act as emulsion stabilizing agents. Unlike surfactant molecules, the cellulose nanocrystals irreversibly adsorb at liquid/liquid interfaces due to their high energy of adsorption, and therefore, the Pickering emulsion is generally a more stable emulsion than that stabilized by surfactants.

### Alternative Starting Materials

It will be apparent to the skilled person that cellulose nanocrystals other than cellulose I nanocrystals as well as microcrystalline cellulose (MCC) can be used as a starting material in the above method to manufacture microparticles.

MCC is a type of fine white, odorless, water-insoluble irregularly shaped granular material. Indeed, MCC particles are basically chunks (i.e. roughly cut pieces) of cellulose microfibrils (which themselves are large bundles of cellulose nanofibrils—see FIG. 1). As such, MCC particles are typically elongated in shape. Furthermore, MCC particles typically exhibit dangling cellulose nanofibrils (or small bundles of nanofibrils). MCC has lower crystallinity than cellulose nanocrystals since the amorphous cellulose regions contained between the crystalline cellulose regions is retained in the MCC and mostly removed in the cellulose nanocrystals.

To make MCC, natural cellulose from wood pulp or cotton linters is first hydrolyzed by combinations of base and acid to obtain hydrocellulose, then bleached and subjected to post-treatment such as grinding and screening processes. MCC typically has a degree of crystallinity of 60% or more, particle sizes of around 20-80 μm, and leveling off degree of polymerization below 350. In some cases, smaller MCC particle sizes can be achieved by special processing. For example, JSR® offers MCC as a 4-micron size granular MCC powder that goes by the trade name Vivapur® CS 4FM. MCC has been widely used in the food, chemical and pharmaceutics industries because of these characteristics.

When using MCC, larger microparticles (compared to particles obtaining from nanocrystals) are typically produced.

### Definitions

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Herein, the notation “% w/v” refers a concentration expressed as the weight of solute in grams per 100 ml of solution. For example, a solution with 1 g of solute dissolved in a final volume of 100 mL of solution would be labeled as “1% m/v”.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary meaning. In embodiments, it may mean plus or minus 10% or plus or minus 5% of the numerical value qualified.

Unless otherwise defined, all technical and scientific terms used herein have the same meaning as commonly understood by one of ordinary skill in the art to which this invention belongs.

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENTS

The present invention is illustrated in further details by the following non-limiting examples.

Calibration Curve for Manufacturing Microparticles with Predetermined Oil Uptake

A calibration curve was first generated to be used interpolate the ratio of nanoemulsion volume to the mass of CNC. This curve was used to predict how much nanoemulsion and CNC were required to produce microparticles with various target oil uptakes. A series of porous microparticles was produced using various nanoemulsion volume to CNC mass ratios. The oil uptake of these microparticles was measured. From these data, a calibration curve was drawn. Then, the calibration curve was used to produce microparticles with desired oil uptakes as reported in Examples 1 to 3 below.

Below, we describe generation of one of the points of the calibration curve (the point corresponding to an oil uptake of 115 mL/100 g). The other points of the calibration curve were gathered in a similar manner using other nanoemulsion volume to CNC mass ratios, which resulted in other oil uptakes.

A nanoemulsion was first prepared as follows: 52.5 mL PEG-25 hydrogenated castor oil (PEG-25 HCO), 52.5 mL Tween 80, and 140 mL alkyl benzoate were poured into a 3.5 L glass beaker. Distilled water was added to the mixture to make the final volume 3.5 L. The mixture was stirred at 700 rpm for 20 min before being separated into 4 1 L bottles and sonicated using a probe sonicator. This was followed by 1.0 h sonication at 60% amplitude (sonics vibra cell) in a water bath to produce 50 nm nanoemulsion by dynamic light scattering.

A CNC+ stock solution of 2 wt % was prepared from PDDA stock solution by diluting 20 wt % PDDA (Mw=400,000 to 500,000) with distilled water. A concentrated CNC suspension was diluted to 1 wt % and then 2 wt % PDDA solution was added to CNC suspension at a solid mass ratio of 14% (PDDA/CNC). The mixture was stirred for 3 min at 1000 rpm before sonication using flow cell with an amplitude of 60%, flow cell pressure of 20-25 psi, stirring rate of 1000 rpm. Sonication time was 2 hr for ˜15 L suspension.

Then, 0.69 L of nanoemulsion was added to 5.7 L CNC+ (0.84 wt %) stock solution with mixing at 400 rpm. After 5 min, 2.03 L CNC (4.53 wt %) stock solution was added and the mixture was stirred for another 15 min before spray-drying. Accordingly, the ratio of nanoemulsion (NE) volume/CNC=690 ml/139.84 g=4.93 ml/g.

For spray drying (SD 1), the outlet temperature was adjusted to 80-95° C. The solids content of the mixture was adjusted to 1.60-2.30 wt. % to ensure smooth spray-drying. The spray drier parameters were as follows: inlet temperature 185C, outlet temperature: 85C, feed stroke 28%, nozzle pressure 1.50 bar, differential pressure 180 mmWc, nozzle air cap 70.

The nanoemulsion was extracted from the microbead powder as follows: 20 g of spray dried ChromaPur OT microbeads was added to 200 mL isopropanol and mixed for 3 min before being centrifuged at 1200 rpm for 6 min. This was repeated, after which the sample was collected, washed and centrifuged and then redispersed into 20 mL isopropanol. The suspension was then poured into a 500 mL evaporating flask and dried in a vacuum of 25 mbar (Heidolph rotary evaporator) at 35° C. with rotation at 70 rpm. A white free-flowing powder was obtained after 2 hours.

The oil uptake was measured to be 115 mL/100 g castor oil. The coordinates for the point on the calibration curve were thus (4.93, 115).

In a similar manner, the remaining points on the calibration curve were obtained for NE/CNC 14.59 (180 g/100 ml oil uptake), and NE/CNC 34.16 (299 g/100 ml oil uptake). The calibration curve was used to predict the oil uptake of microparticles depending on their manufacturing conditions. More specifically, as shown in Examples 1 to 3, the calibration curve was used to calculate how much nanoemulsion and cCNC+ must be combined to achieve a desired oil uptake.

Notwithstanding the method to generate a calibration curve for a nanoemulsion, one can also generate a calibration curve for a microemulsion.

### Materials & Methods

Sodium Carboxylate Nanocrystalline Cellulose (cCNC) and cCNC Stock Suspension

Sodium carboxylate nanocrystalline cellulose (cCNC) was produced as described in International patent publication no. WO 2016\015148 A1. Briefly, dissolving pulp (Temalfa 93) is dissolved in 30% aqueous hydrogen peroxide and heated to reflux with vigorous stirring over a period of 8 hours. The resulting suspension is diluted with water, purified by diafiltration and then neutralized with aqueous sodium hydroxide.

As produced from the reaction of 30% aqueous hydrogen peroxide with dissolving pulp, a concentrated stock suspension of sodium carboxylate nanocrystalline cellulose (cCNC) typically consisted of 4% CNC in distilled water. This stock suspension was diluted with distilled water as needed for use in the Examples below.

Cationic cCNC (i.e. cCNC+) Stock Suspension

A PDDA (polydiallyldimethylammonium chloride; CAS: 26062-79-3) solution was prepared by diluting a 20 wt % solution of PDDA (Mw=400,000 to 500,000) with distilled water to prepare stock solutions of 2 wt %.

The above concentrated sodium carboxylate CNC suspension was diluted to 1 wt %. Then, the 2 wt % PDDA solution was added to the carboxylate salt of CNC (cCNC) suspension at a solid mass ratio of 14% (PDDA/cCNC). The mixture was stirred for 3 min at 1000 rpm before sonication using flow cell with an amplitude of 60%, flow cell pressure of 20-25 psi, stirring rate of 1000 rpm. The resulting cationic cCNC+ suspension was purified by diafiltration (Diafiltration unit (Spectrum Labs, KrosFlo TFF System)).

This cCNC+ stock suspension was diluted with distilled water as needed for use in the Examples below.

### Nanoemulsion A Preparation

52.5 mL PEG-25 hydrogenated castor oil (Croduret™ 25—CAS: 61788-85-0), 52.5 mL Tween 80 (Polysorbate 80-Lotioncrafter—CAS 9005-65-6), and 140 mL alkyl benzoate (C₁₂-C₁₅ Alkyl Benzoate, Lotioncrafter Ester AB—CAS: 68411-27-8) were poured into a 3.5 L glass beaker. Distilled water was added to the mixture to make the final volume 3.5 L. The mixture was stirred at 700 rpm for 20 min (VMI Rayneri Turbotest mixer equipped with a saw tooth blade). The mixture was then subjected to 1.0h sonication at 60% amplitude (sonics vibra cell) cooled in water bath to produce a nanoemulsion that appeared translucent, with a slight blue tinge. After sonication, the nanoemulsion size was measured to be 45-50 nm by dynamic light scattering (NanoBrook 90 Plus, Brookhaven Instruments).

### Spray-Drying

A model SD 1 spray dryer (Techni Process) was used to produce the microparticles as described below. Specific parameters used in spray drying are provided in the Examples.

### Characterization

Particle size and particle size distribution were analyzed using particle size analyzer (Sysmex FPIA-3000).

Oil uptake was measured using the fluid saturation method as described in US standard ASTM D281-84. Water uptake was measured using the fluid saturation method as described in US standard ASTM D281.

The surface area was measured using the BET (Brunauer-Emmett-Teller) method as described above.

Scanning electron microscopy images (SEM) images were obtained on uncoated samples with an FEI Inspect F50 FE-SEM at 2.00 kV.

### Example 1—Microparticles Produced with a Nanoemulsion/CNC Ratio of 4.64 Ml/Gram

0.73 wt % cCNC+ and 3.91 wt % cCNC suspensions were prepared from the above stock suspensions.

0.85 L of nanoemulsion A was added to 8.5 L of the CNC+ suspension with mixing at 800 rpm. After 5 min, 3.1 L of cCNC (3.91 wt %) suspension were added and the mixture was stirred for another 30 min before spray-drying. Additional 3 L water was added to the mixture to allow the sample to be spray-dried easily.

The spray drier parameters were set as follows: inlet temperature 185C, outlet temperature: 85C, feed stroke 28%, nozzle pressure 1.50 bar, differential pressure 180 mmWc, nozzle air cap 70. The process yielded a dried free-flowing white powder.

To remove the embedded porogen, a 20 g lot of the spray dried microparticles was added to 200 mL isopropanol and mixed for 3 min before being centrifuged at 1200 rpm for 6 min. This step was repeated one time, discarding the supernatant liquid each time. The sample was then dispersed into 20 mL isopropanol. The dispersion was poured into a 500 mL evaporating flask and dried in a vacuum of 25 mbar (Heidolph rotary evaporator; (Basis Hei-Vap ML)) at 35° C. with rotation at 70 rpm.

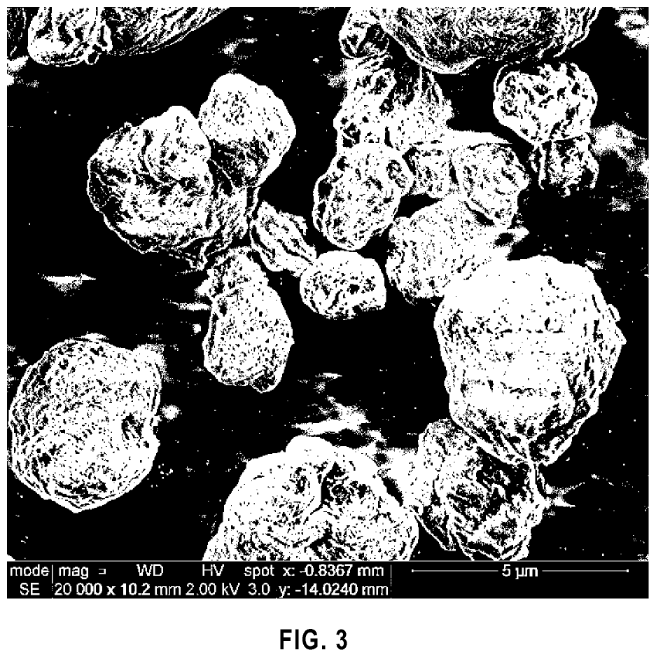

A white free-flowing powder was formed after 2 hours drying. Its properties are summarized in Table 1 below. A typical SEM image is shown in FIG. 3.

### Example 2—Microparticles Produced with a Nanoemulsion/CNC Ratio of 14.49 Ml/Gram

0.84 wt % cCNC+ and 4.53 wt % cCNC suspensions were prepared from the above stock suspensions.

2.6 L of Nanoemulsion A was added to 7.2 L CNC+ (0.84 wt %) suspension with mixing at 400 rpm. After 5 min, 2.6 L cCNC (4.53 wt %) suspension were added and the mixture was stirred for another 5 min before spray-drying. The mixture was found to be very viscous, so the solid content concentration was reduced as follows: 2.2 L distilled water was added to the mixture above (12.4 L) to give a final mixture of 14.6 L.

The spray drier parameters were the same as in Example 1. The process yielded a dried free-flowing white powder. The porogen removal and the isolation/drying of the product were as described in Example 1.

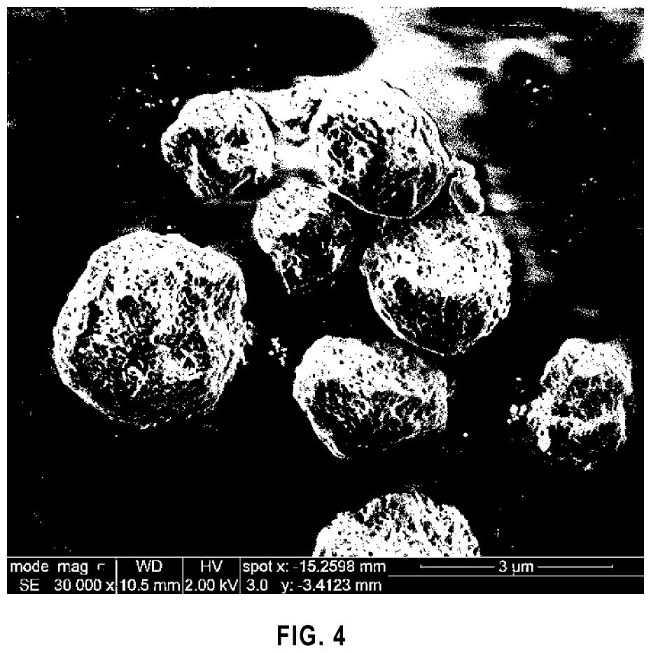

A white free-flowing powder was formed after 2 hour drying. Its properties are summarized in Table 1 below. A typical SEM image of the powder is shown in FIG. 4.

### Example 3—Microparticles Produced with a Nanoemulsion/CNC Ratio of 29.11 Ml/Gram

0.84 wt % cCNC+ and 4.53 wt % CNC suspensions were prepared from the above stock suspensions.

2.8 L of Nanoemulsion A was added to 3.9 L cCNC+(0.84 wt %) suspension with mixing at 400 rpm. After 5 min, 1.4 L cCNC (4.53 wt %) suspension were added and the mixture was stirred for another 5 min before spray-drying.

The spray drier parameters were the same as in Example 1. The process yielded a dried free-flowing white powder. The porogen removal and the isolation/drying of the product were as described in Example 1.

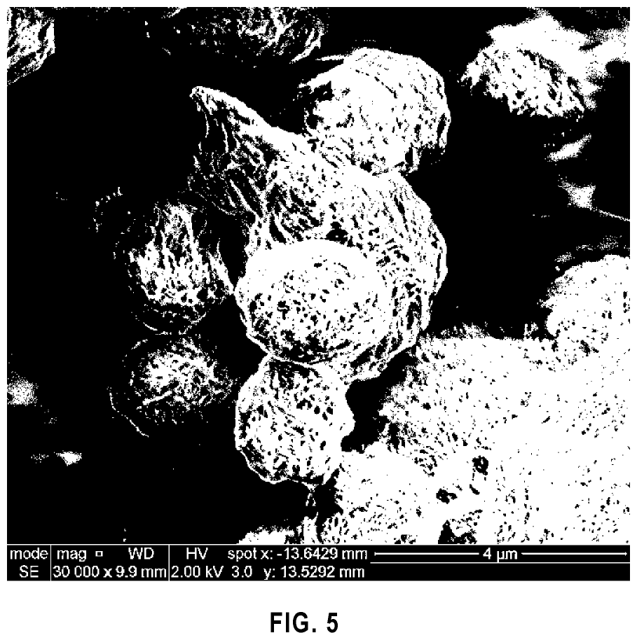

A white free-flowing powder was formed after 2 hours. Its properties are summarized in Table 1 below. A typical SEM image of the powder is shown in FIG. 5.

### Comparative Example 1—Microparticles Produced without Emulsion

For comparison, microparticles were produced by spray-drying a CNC suspension that did not contain any nanoemulsion as taught in International patent publication no. WO 2016\015148 A1.

A 4 wt % CNC suspension was prepared. The suspension was spray dried under the same conditions described in Example 1. The process yielded a dried free-flowing white powder. The powder exhibited a size range of 2.1-8.7 μm. The oil uptake was 55 ml/100 g. Other data are listed in Table 1.

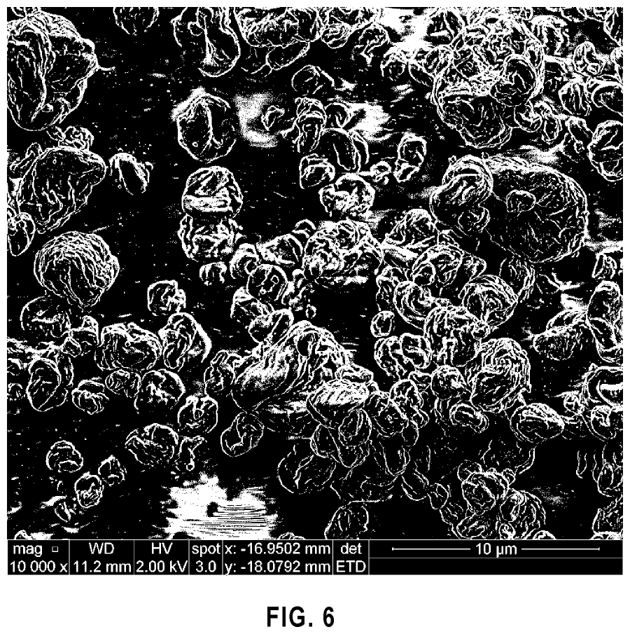

A typical SEM image of the powder is shown in FIG. 6.

### Characterization of the Microparticles of Examples 1-3 and Comp. Ex. 1

Table 1 collects oil uptake and other physical data for cellulose microparticles made from a nanoemulsion, followed by extraction of the nanoemulsion constituents (Examples 1 to 3) as well as comparative Example 1, which is a control made from CNC without the use of a nanoemulsion. The ratio of the volume of nanoemulsion (ml) to the total weight of CNC (g) used for preparing the microparticles is also reported.

Increased oil uptake correlates with increased water uptake and increased surface area. Increased oil uptake correlates inversely with bulk density and refractive index.

| | Comparative | | | |
|---|---|---|---|---|
| | Example 1 | Example 1 | Example 2 | Example 3 |
| Nanoemulsion volume/weight CNC (ml/g) | N/A | 4.64 | 14.49 | 29.11 |
| Castor oil uptake (ml/100 g) | 55 | 108 | 172 | 252 |
| Water uptake (ml/100 g) | 105 | 132 | 184 | 236 |
| Average particle size D₅₀ (μm) | 10 | 8 | 9 | 12 |
| Size distribution D₁₀/D₉₀ (μm) | 5/19 | 5.1/15.0 | 5/15 | 5/25 |
| Bulk density (g/cm³) | 0.53 | 0.32 | 0.23 | 0.15 |
| Surface area (m²/g) | 15 | 86 | 157 | 168 |
| Appearance | White powder | White powder | White powder | White powder |
| pH | 5 | 5 | 5 | 5 |
| Refractive index | 1.54 | 1.49 | 1.45 | 1.45 |

It can be observed that the refractive index of the microparticles decreases as the oil uptake and surface area increase.

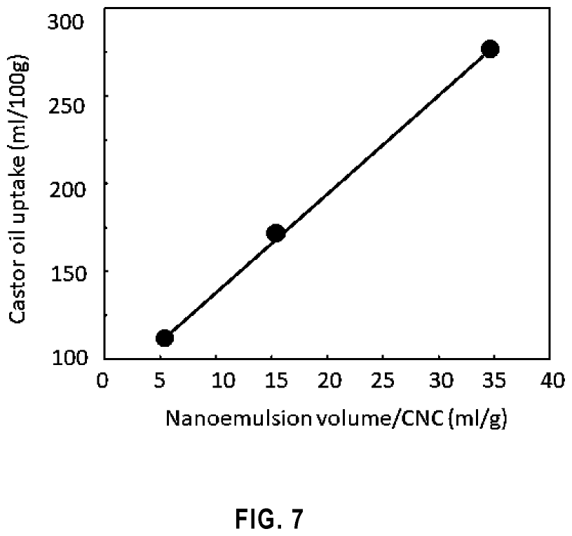

As can be seen from Table 1, the oil uptake of the microbead increases with the ratio of the volume of nanoemulsion (ml) to the total weight of CNC (g) used for preparing the microparticles. In fact, when these data are plotted, see FIG. 7, a linear correlation is clearly observed.

### Mattifying Effect of the Microparticles of Examples 1-3

The mattifying effect of the microparticles Examples 1-3 and Comparative Example 1 was measured and compared to that of various conventional cellulose-based products—see FIG. 8. The mattifying effect was determined as % reflectivity. More specifically, the matte effect is determined through the equation Rₘₐₜₜₑ(%)=100(R_{Diffuse}/Rₜₒₜₐₗ). In this equation, Rₘₐₜₜₑ is the matte reflectance, R_{Diffuse} is the diffuse reflectance and Rₜₒₜₐₗ is the total reflectance. Measurements of the quantities were obtained by means of a Seelab GP 150 spectrometer.

The mattifying effect of a control sample of an oil-in-water emulsion with no added microbeads is also shown. It is evident from FIG. 8 that porous cellulose microparticles of Examples 1 exhibit a better matte effect than all other cellulose-based materials except for Vivapur®. Nevertheless, the microparticles of Examples 2 and 3 also outperform Vivapur® in terms of matte effect.

The conventional products were biobased products developed/sold for cosmetic applications. These were:
Vivapur® CS9 FM: microcrystalline cellulose (which is not in the form microparticles) sold by JRS Pharma®;
Rice PO₄ Natural®: phosphate crosslinked rice starch for application in cosmetics, CAS 55963-33-2, sold by Agrana Starch®;
Tego® Feel Green: 100% natural microcrystalline cellulose cosmetic powder (which is not in the form microparticles), 6-10 μm average particle size, sold by Evonik® Industries;
Cellulobeads® D5 and D10, respectively 5 and 10 μm spherical cellulose beads derived from the viscose process, followed by emulsion precipitation—for cosmetic applications sold by Daito Kasei®;
Celluloflake, cellulose flakes for cosmetic applications sold by Daito Kasei®; and
Avicel® PC 106 sold by FMC Biopolymers®: 20 μm size microcrystalline cellulose white to yellowish brown free flowing powder (which is not in the form microparticles).

We noted that, because of their manufacturing method, Avicel® products, Tego® Feel C10, and Vivapur® CS9 FM each have an oil uptake that is fixed (i.e. not tunable), which is less desirable for the cosmetics industry. Daito Kasei's Cellulobeads are made by the viscose process. Hence, they offer a certain degree of oil uptake, but the oil uptake range is limited by the fact that their manufacturing method cannot be adapted to obtain various particles with different oil uptake.

### Skin Feel of the Microparticles of Examples 1-3

The skin feel of the microparticles of Examples 1-3 and compared to that of the above various conventional cellulose-based products. A sensorial panel of experts was used for this purpose.

Compared with Avicel® products (such as PH 101, 50 μm particle size) sold by FMC Biopolymers®, Tego® Feel Green sold by Evonik® Industries, or Vivapur® Sensory 5 (5 μm particle size) and Sensory 15S (15 μm particle size) sold by JRS Pharma®, the microparticles of Examples 1-3 had better feel to the skin.

### Example 4—Microparticles Produced with a Self-Extracting Limonene Nanoemulsion

3 mL PEG-25 hydrogenated Castor Oil (Croduret™ 25—CAS: 61788-85-0), 3 mL Tween 80 (Polysorbate 80-Lotioncrafter—CAS 9005-65-6), 12 mL limonene ((R)-(±)-Limonene (Sigma-Aldrich—CAS: 5989-27-5)), and 180 mL distilled H₂O were poured into a 0.25 L nalgene bottle and sonicated using the probe sonicator for 30 minutes at 60% amplitude (sonics vibra cell VCX) in water bath to produce an emulsion. After sonication, emulsion size was measured by dynamic light scattering to be ˜20 nm.

Chitosan stock solution (1 wt %) was prepared by dissolving 10 g chitosan in 1000 mL of 0.1M HCL. 700 mL of the 1 wt % chitosan solution (7 g) was added to 5000 mL of a 1% cCNC suspension (50 g). The cCNC+ mixture was stirred for 3 minutes at 1000 rpm before sonication using probe equipped with a flow cell with an amplitude of 60%, flow cell pressure of 20-25 psi, and a flow rate of 2 L/min for 2 hours. The slurry was purified by diafiltration using a 70 kDa MW cut-off hollow fiber filter until a permeate conductivity of 50 μs and pH of 5 was reached. The slurry was then concentrated to 1% w/v yielding a stable, viscous suspension of positively charged particles.

0.20 L limonene nanoemulsion was added to 0.56 L cCNC+(0.81 wt %) stock solution with mixing at 400 rpm. After 5 min, 0.20 L CNC (4.4 wt %) stock solution was added and the mixture was stirred for another 15 min. Solids content of the mixture was adjusted to 1.60 wt. % to ensure smooth spray-drying.

The slurry was then spray dried using an SD-1 spray dryer (Techni Process) using an inlet temperature of 210° C. with an outlet temperature of 85° C. Compressed air pressure was set to 1.5 bar, with a feed rate of approximately 3 L/min to the dryer.

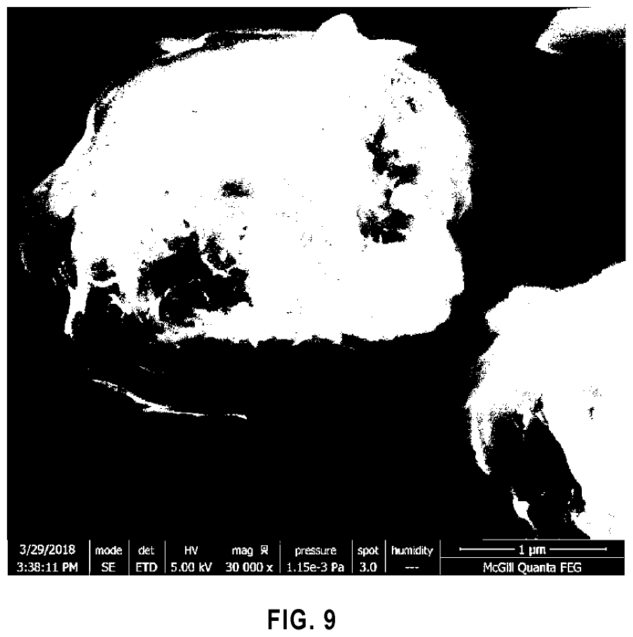

The oil uptake of spray dried microparticles was found to be 100 mL castor oil/100 g. The microparticles were imaged under scanning electron microscope and pores with a size of ˜100 nm were observed on the surface of microparticles—see FIG. 9.

### Example 5—Microparticles Produced with a Self-Extracting Pinene/Methylcellulose Macroemulsion

A self-extracting macroemulsion was made as follows: 1 g methyl cellulose (Sigma-Aldrich—CAS: 9004-67-5; Mw: 41,000 Da) was added to 500 mL distilled water and stirred for 6 h to ensure complete dissolution. 40 mL α-Pinene (Sigma-Aldrich—CAS: 80-56-8) was then poured into the methyl cellulose solution and stirred at 500 rpm for 10 min. The mixture was then sonicated using a probe sonicator for 30 minutes at 60% amplitude (sonics vibra cell VCX) in a water bath to produce the emulsion. After sonication, emulsion size was measured by dynamic light scattering to be approximately 1.5 μm.

A chitosan stock solution (1 wt %) was prepared by dissolving 10 g chitosan (Sigma-Aldrich—CAS: 9012-76-4, Mw: 50,000-190,000 Da) in 1000 mL of 0.1M HCL. 700 mL of the 1 wt % chitosan solution (7 g) was added to 5000 mL of a 1% CNC suspension (50 g). The mixture was stirred for 3 minutes at 1000 rpm before sonication using probe equipped with a flow cell with an amplitude of 60%, flow cell pressure of 20-25 psi, and a flow rate of 2 L/min for 2 hours. The slurry was purified by diafiltration using a 70 kDa MW cut-off hollow fiber filter until a permeate conductivity of 50 μs and pH of 5 was reached. The slurry was then concentrated to 1% w/v yielding a stable, viscous suspension of positively charged particles.

0.51 L methylcellulose/pinene macroemulsion was added to 0.25 L cCNC+ (0.73 wt %) stock solution with mixing at 400 rpm. After 5 min, 0.20 L cCNC (3.5 wt %) stock solution was added and the mixture was stirred for another 15 min. Solids content of the mixture was adjusted to 1.60 wt. % to ensure smooth spray-drying.

The slurry was then spray dried using an SD-1 spray dryer (Techni Process) using an inlet temperature of 210° C. with an outlet temperature of 85° C. Compressed air pressure was set to 1.5 bar, with a feed rate of approximately 3 L/min to the dryer.

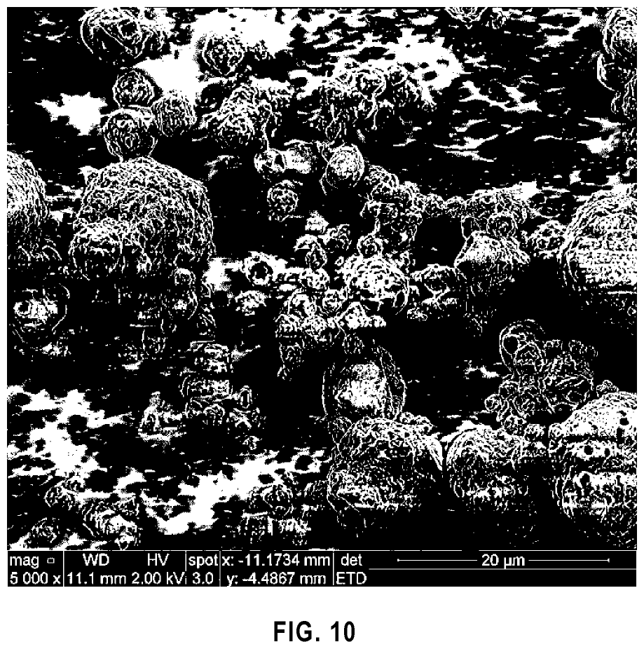

The oil uptake of spray dried microparticles was found to be 160 mL castor oil/100 g. The microparticles were imaged under scanning electron microscope and pores with a size of ˜1 micron were observed on the surface of microparticles—see FIG. 10.

### Example 6—Microparticles Produced with a Self-Extracting α-Pinene/Gelatin Macroemulsion

A self-extracting macroemulsion was made as follows: 2.5 g gelatin was added to 500 mL distilled water and stirred for 6 h to ensure complete dissolution. 40 mL pinene was then poured into the gelatin solution and stirred at 500 rpm for 10 min. The mixture was then sonicated using the probe sonicator for 30 minutes at 60% amplitude (sonics vibra cell VCX) in water bath to produce emulsions. After sonication, emulsion size was measured by dynamic light scattering to be ˜1.1 μm.

Chitosan stock solution (1 wt %) was prepared by dissolving 10 g chitosan in 1000 mL of 0.1M HCL. 700 mL of the 1 wt % chitosan solution (7 g) was added to 5000 mL of a 1% cCNC suspension (50 g). The cCNC+ mixture was stirred for 3 minutes at 1000 rpm before sonication using probe equipped with a flow cell with an amplitude of 60%, flow cell pressure of 20-25 psi, and a flow rate of 2 L/min for 2 hours. The slurry was purified by diafiltration using a 70 kDa MW cut-off hollow fiber filter until a permeate conductivity of 50 μs and pH of 5 was reached. The slurry was then concentrated to 1% w/v yielding a stable, viscous suspension of positively charged particles.

0.52 L gelatin/pinene macroemulsion was added to 0.47 L cCNC+ (0.73 wt %) stock solution with mixing at 400 rpm. After 5 min, 0.22 L CNC (3.5 wt %) stock solution was added and the mixture was stirred for another 15 min. Solids content of the mixture was adjusted to 1.60 wt. % to ensure smooth spray-drying.

The slurry was then spray dried using an SD-1 spray dryer (Techni Process) using an inlet temperature of 210° C. with an outlet temperature of 85° C. Compressed air pressure was set to 1.5 bar, with a feed rate of approximately 3 L/min to the dryer.

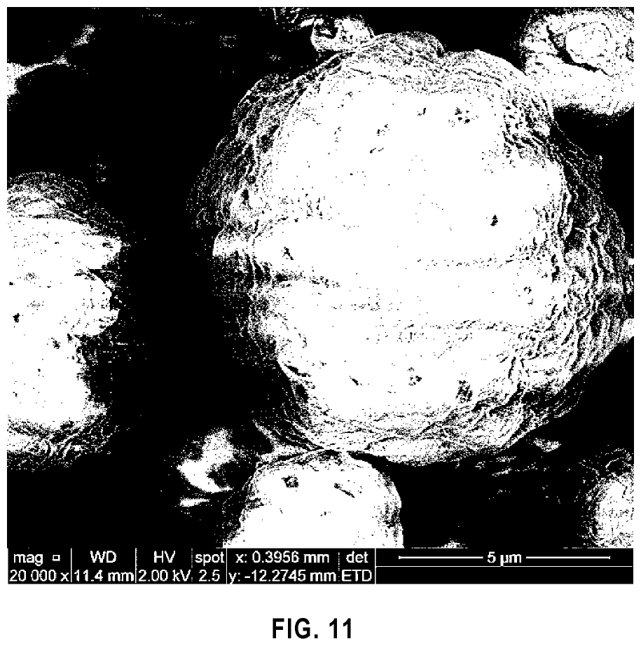

The oil uptake of spray dried microparticles was found to be 210 mL castor oil/100 g. The microparticles were imaged under scanning electron microscope and pores with a size of ˜1 micron were observed on the surface of microparticles—see FIG. 11.

### Example 7—Microparticles Produced with a Self-Extracting α-Pinene/MONTANOV™ Macroemulsion

A self-extracting macroemulsion was made as follows: 1 g MONTANOV™ 82 (INCI: Cetearyl Alcohol and Coco-Glucoside) was added to 500 mL distilled water and stirred for 6 h to ensure complete dissolution. 40 mL pinene was then poured into the MONTANOV™ 82 solution and mixed at 500 rpm for 10 min. The mixture was then sonicated using the probe sonicator for 30 minutes at 60% amplitude (sonics vibra cell VCX) in a water bath to produce the emulsion. After sonication, the emulsion size was measured by dynamic light scattering to be ˜0.5 μm.

No polyelectrolyte was added to the stock cCNC suspension.

0.54 L MONTANOV™ 82/pinene macroemulsion was added to 0.24 L cCNC (4.22 wt %) stock solution. An additional 150 mL of distilled water was added, and the suspension was then mixed at 800 rpm for 15 minutes. Solids content of the mixture was adjusted to 1.60 wt. % to ensure smooth spray-drying.

The slurry was then spray dried using an SD-1 spray dryer (Techni Process) using an inlet temperature of 210° C. with an outlet temperature of 85° C. Compressed air pressure was set to 1.5 bar, with a feed rate of approximately 3 L/min to the dryer.

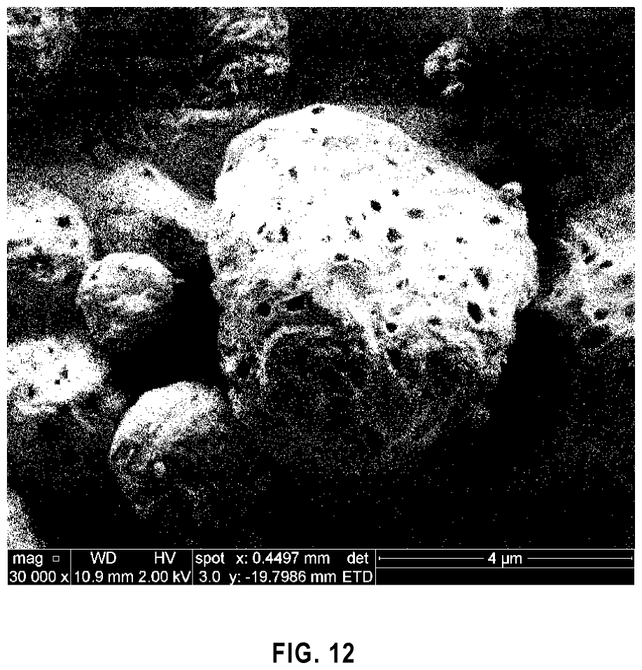

The oil uptake of spray dried microparticles was found to be 290 mL corn oil/100 g. A typical SEM image of the powder is shown in FIG. 12.

### Example 8—Microparticles Produced with a Self-Extracting α-Pinene/SEPIFEEL™ Macroemulsion

A self-extracting macroemulsion was made as follows: 1 g SEPIFEEL™ ONE (INCI: Palmitoyl Proline & Magnesium Palmitoyl Glutamate & Sodium Palmitoyl Sarcosinate) was added to 500 mL distilled water and stirred for 6 h to ensure complete dissolution. 40 mL pinene was then poured into the SEPIFEEL™ ONE solution and mixed at 800 rpm for 10 min. The mixture was then sonicated in a cooling water bath using a probe sonicator for 30 minutes at 60% amplitude (sonics vibra cell VCX). After sonication, the emulsion size was measured by dynamic light scattering to be ˜0.6 μm.

No polyelectrolyte was added to the stock cCNC suspension.

cCNC 0.54 L SEPIFEEL™ ONE/pinene macroemulsion was added to 0.24 L CNC (4.22 wt %) stock solution. An additional 150 mL of distilled water was then added. The suspension was mixed at 800 rpm. After 15 min of mixing, the slurry was then spray dried using an SD-1 spray dryer (Techni Process) using an inlet temperature of 210° C. with an outlet temperature of 85° C. Compressed air pressure was set to 1.5 bar, with a feed rate of approximately 3 L/min to the dryer. The solids content of the mixture was adjusted to 1.60 wt. % to ensure smooth spray-drying.

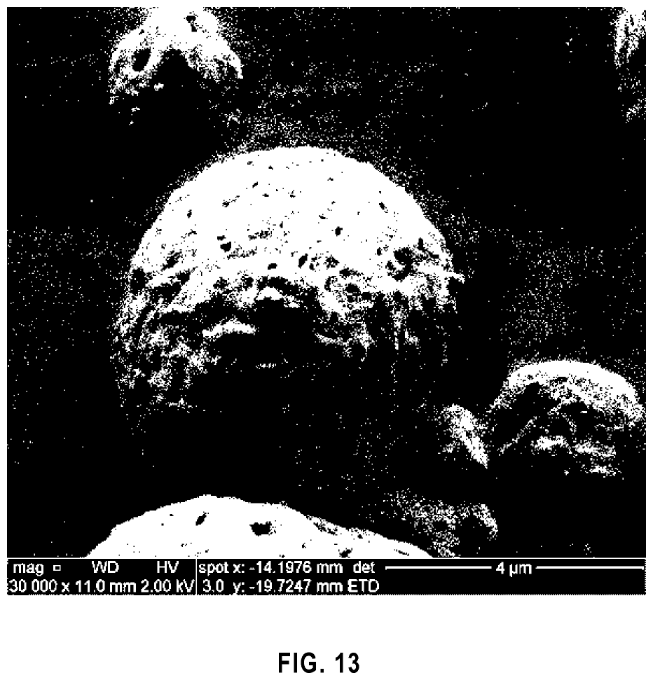

The oil uptake of spray dried microparticles was found to be 320 mL corn oil/100 g. A typical SEM image of the powder is shown in FIG. 13.

### Example 9—Lipophilic Microparticles Produced with a Montanov™ 82 and Alkyl Benzoate Nanoemulsion and with Silk Fibroin

A 400 nm nanoemulsion was prepared as follows: 0.021 g Montanov™ 82 (SEPPIC) was dissolved in 470 ml distilled water at 60° C. 10 g alkyl benzoate was then poured into the Montanov solution and stirred at 60° C. for 10 min at 1000 rpm. The mixture was then sonicated at 60% amplitude (Sonics® Vibra-Cell®) in an iced water bath for 20 min to produce a nanoemulsion with an average droplet diameter of 400 nm. 300 mL NCC suspension (1.90 wt %) was poured into the above emulsion and mixed at 300 rpm for 10 min.

1-2 g of silk fibroin (from Ikeda Corporation) was added to 5.55 g CaCl₂), 4.6 g ethanol, 7.2 g distilled water (molar ratio of CaCl₂:Ethanol:H₂O was 1:2:8) at 80° C. (Caution: this “Ajisawa” solvent mixture generates a lot of heat). Silk fibroin was pressed down so it was fully immersed in the solvent. After 20-30 min, the fibroin seemed completely dissolved and the solution became transparent with a tint of yellow color. The fibroin solution was pipetted to a cellulose dialysis tube and dialysed against distilled water in a 3.5 L glass beaker. The water was changed every hour for the first day and then changed every half a day. The whole dialysis process took three days. The concentration of the solution in the dialysis tube after dialysis was 1.5-2.0 wt %.

28 ml of the above fibroin solution (1.88 wt %) were poured into the above CNC/nanoemulsion mixture and stirred at 300 rpm for 10 min before spray-drying (inlet temperature 185° C., outlet temperature: 85° C., feed stroke 28%, nozzle pressure 1.50 bar, differential pressure 180 mmWc, nozzle air cap 70). The process yielded a dried free-flowing white powder.

To remove the embedded porogen and induce fibroin β-sheet formation, a 2 g lot of the spray dried microbeads was added to 40 mL ethanol and mixed for 3 min before being centrifuged at 1200 rpm for 6 min. This step was repeated one time, discarding the supernatant liquid each time. The sample was then dispersed into 20 mL ethanol. The dispersion was poured into a 500 mL evaporating flask and dried in a vacuum of 25 mbar (Heidolph rotary evaporator; (Basis Hei-Vap ML)) at 60° C. with rotation at 70 rpm. A white free-flowing powder was formed after 1 hour.

The powder did not mix well with water and stayed on the surface of water level when added to water. The oil uptake was measured to be 195 ml/100 g.

### Example 10—Lipophilic Microparticles Produced with a Montanov™ 82 and Alpha-Pinene Nanoemulsion and with Silk Fibroin

A 900 nm nanoemulsion was prepared as follows: 0.021 g Montanov™ 82 (SEPPIC) was dissolved in 470 ml distilled water at 60° C. 10 g alpha-pinene was then poured into Montanov solution and stirred at 60° C. for 10 min at 1000 rpm. The mixture was then sonicated at 60% amplitude (Sonics® Vibra-Cell®) in an iced water bath for 20 min to produce an emulsion with an average diameter of 900 nm. 300 mL cNCC suspension (1.90 wt %) was poured into the above emulsion and mixed at 300 rpm for 10 min.

23 ml fibroin solution (1.88 wt %), prepared according to Example 9, was poured into the above mixture and stirred at 300 rpm for 10 min before spray-drying (inlet temperature 210° C., outlet temperature: 85° C., feed stroke 28%, nozzle pressure 1.50 bar, differential pressure 180 mmWc, nozzle air cap 70). The process yielded a dried free-flowing white powder.

The powder did not mix well with water and stayed on the surface of water level when added to water. The oil uptake was measured to be 105 ml/100 g.

### Example 11—Hydrophilic Microparticles Produced with a Montanov™ 82 (in Excess) and Alpha-Pinene Nanoemulsion and with Silk Fibroin

A 840 nm nanoemulsion was prepared as follows: 0.500 g Montanov™ 82 (SEPPIC) was dissolved in 350 ml distilled water at 60° C. 20 g alpha-pinene was then poured into Montanov solution and stirred at 60° C. for 15 min at 1000 rpm. The mixture was then sonicated at 60% amplitude (Sonics® Vibra-Cell®) in iced water bath for 15 min to produce emulsions with an average diameter of 840 nm. 466 mL cCNC suspension (2.16 wt %) was poured into the above emulsion and mixed at 300 rpm for 10 min.

12.7 ml fibroin solution (1.59 wt %), prepared according to Example 9, was poured into the above mixture and stirred at 300 rpm for 10 min before spray-drying. The spray drier parameters were set as follows: inlet temperature 210° C., outlet temperature: 85° C., feed stroke 28%, nozzle pressure 1.50 bar, differential pressure 180 mmWc, nozzle air cap 70. The process yielded a dried free-flowing white powder.

The powder sank quickly to the bottom of water once added to water. The oil uptake was measured to be 185 ml/100 g.

### Example 12—Microparticles Produced with a Self-Extracting α-Pinene/SEPIFEEL™ Macroemulsion and a Low Concentration of Cationic Starch

This Example shows that cationic starch can be used in place of chitosan or polydiallyldimethylammonium chloride.

1 g SEPIFEEL™ ONE (INCI: Palmitoyl Proline & Magnesium Palmitoyl Glutamate & Sodium Palmitoyl Sarcosinate) was added to 450 mL distilled water and stirred for 1 h at 90° C. to ensure complete dissolution. 43 g α-pinene was then poured into the SEPIFEEL™ ONE solution and stirred at 1000 rpm for 15 min. The mixture was then sonicated using a probe sonicator (sonics vibra cell VCX) for 30 min at 60% amplitude in water bath to produce the emulsion. After sonication, the emulsion size was measured DLS to be ˜0.6 μm.

Cationic starch (INCI: starch hydroxypropyltrimonium chloride, Roquette, HI-CAT 5283A) stock solution (1 wt %) was prepared by dissolving 10 g cationic starch in 990 mL of distilled water at 90° C. 60 g 1 wt % cationic starch solution was added to 528 g CNC suspension (3.79 wt %) and mixed for 30 min at 400 rpm. Then the emulsion (500 mL) was added and stirred for another 10 min at 400 rpm.

The resulting slurry was spray dried with the following characteristics: inlet temperature 185° C., outlet temperature 85° C., feed stroke 28%, nozzle pressure 1.50 bar, differential pressure 180 mmWc, nozzle air cap 70. Free-flowing spray-dried powder (˜10 g) was then collected and mixed with 80 mL ethanol for 10 min before being centrifuged at 2000 rpm for 6 min. The slurry on the bottom of centrifuge tube was collected at dried on moisture balance (130° C.) for about 30 min. Alternatively, after mixing with ethanol, the slurry was dried on Heidolph rotary evaporator at 20 mbar and 60° C. for 2 hr. The powder was then sieved (150 μm) and heated at 90° C. for an hour.

Minimum cationic starch: To avoid incompatibility with cosmetic formulations due to the presence of positively charged groups, the amount of cationic starch used in the mixture was minimized. The washed and dried porous microbeads were added to distilled water at 3 wt % and vortexed at 500 rpm for 20 seconds. The supernatant was collected one day later and measured using dynamic light scattering. It was found that as we decreased cationic starch/CNC mass ratio from 4% to 3%, the size of disintegrated particle in the supernatant decreased from 640 nm to 550 nm. Thus, it is established that the minimum amount of cationic starch/CNC is 3% for optimum water stability of these microbeads and formulation compatibility.

Properties of the microbeads prepared were as follows.

| | |
|---|---|
| Nanoemulsion volume/weight CNC (ml/g) | 24.98 |
| Castor oil uptake (ml/100 g) | 215 |
| Water uptake (ml/100 g) | 208 |
| Average particle size D₅₀ (μm) | 10.7 |
| Size distribution D₁₀/D₉₀ (μm) | 5.5/19.1 |
| Bulk density (g/cm³) | 0.17 |
| Surface area (m²/g) | N/A |
| Appearance | White powder |
| pH | 5 |
| Refractive index | N/A |

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
International patent publication no. WO 2011/072365 A1
International patent publication no. WO 2013/000074 A1
International patent publication no. WO 20161015148 A1
International patent publication no. WO 2017\101103 A1
US patent publication no. 2005/0255135 A1
Journal of the American Chemical Society, Vol. 60, p. 309, 1938
Habibi et al. 2010, Chemical Reviews, 110, 3479-3500
Okuyama et al., Progress in developing spray-drying methods for the production of controlled morphology particles: From the nanometer to submicrometer size ranges, Advanced Powder Technology 22 (2011) 1-19.

1. Porous cellulose microparticles comprising:
cellulose I nanocrystals aggregated together, thus forming the microparticles, and arranged around cavities in the microparticles, thus defining pores in the microparticles.

2. The microparticles of claim 1, wherein the microporous particles have a castor oil uptake of about 60 ml/100 g or more.

3. (canceled)

4. The microparticles of claim 1, wherein the microporous particles have a surface area of about 30 m²/g or more.

5. (canceled)

6. (canceled)

7. (canceled)

8. (canceled)

9. (canceled)

10. (canceled)

11. (canceled)

12. The microparticles of claim 1, wherein the pores are from about 10 nm to about 500 nm in size.

13. (canceled)

14. (canceled)

15. (canceled)

16. (canceled)

17. (canceled)

18. (canceled)

19. (canceled)

20. The microparticles of claim 1, wherein the cellulose I nanocrystals in the microparticles are carboxylated cellulose I nanocrystals and salts thereof, preferably carboxylated cellulose I nanocrystals or cellulose I sodium carboxylate salt.

21. The microparticles of claim 1, comprising one or more further components in addition to cellulose I nanocrystals and wherein the one or more further components are coated on the cellulose I nanocrystals, deposited on the walls of the pores in the microparticles, or interspersed among the nanocrystals.

22. (canceled)

23. (canceled)

24. The microparticles of claim 21, wherein the cellulose I nanocrystals are coated with a polyelectrolyte layer, or a stack of polyelectrolyte layers with alternating charges.

25. The microparticles of claim 21, wherein the cellulose I nanocrystals are coated with one or more dyes and the one or more dyes are located:
directly on the surface of the cellulose I nanocrystals or
on top of a polyelectrolyte layer, or a stack of polyelectrolyte layers with alternating charges.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. The microparticles of claim 21, wherein a chitosan, a starch, methylcellulose, gelatin, alginate, albumin, gliadin, pullulan, and/or dextran are deposited on the walls of the pores in the microparticles.

39. (canceled)

40. The microparticles of claim 21, wherein a protein, such as silk fibroin or gelatin is interspersed among the nanocrystals.

41. A cosmetic preparation comprising the microparticles of claim 1 and one or more cosmetically acceptable ingredients.

42. (canceled)

43. (canceled)

44. (canceled)

45. (canceled)

46. (canceled)

47. (canceled)

48. (canceled)

49. A method for producing the porous cellulose microparticles of claim 1, the method comprising the steps of:
a) providing a suspension of cellulose I nanocrystals;
b) providing an emulsion of a porogen,
c) mixing the suspension with the emulsion to produce a mixture comprising a continuous liquid phase in which droplets of the porogen are dispersed and in which the nanocrystals are suspended;
d) spray-drying the mixture to produce microparticles; and
e) if the porogen has not sufficiently evaporated during spray-drying to form pores in the microparticles, evaporating the porogen or leaching the porogen out of the microparticles to form pores in the microparticles.

50. The method of claim 49, further comprising the steps of:
establishing a calibration curve of the porosity or the oil uptake of the microparticles to be produced as a function of the emulsion volume to cellulose I nanocrystals mass ratio of the mixture of step c),
using the calibration curve to determine the emulsion volume to cellulose I nanocrystals mass ratio of the mixture of step c) allowing to produce microparticles with a desired porosity or a desired oil uptake, and
adjusting the emulsion volume to cellulose I nanocrystals mass ratio of the mixture of step c) in order to produce microparticles with the desired porosity or the desired the oil uptake.

51. (canceled)

52. (canceled)

53. (canceled)

54. (canceled)

55. (canceled)

56. The method of claim 49, wherein a liquid phase of the suspension in step a) is water or a mixture of water with one or more water-miscible solvent.

57. The method of claim 56, wherein the water-miscible solvent is acetaldehyde, acetic acid, acetone, acetonitrile, 1,2-, 1,3-, and 1,4-butanediol, 2-butoxyethanol, butyric acid, diethanolamine, diethylenetriamine, dimethylformamide, diemthoxyethane, dimethylsufoxide, ethanol, ethyl amine, ethylene glycol, formic acid, fufuryl alcohol, glycerol, methanol, methanolamine, methyldiethanolamine, N-methyl-2-pyrrolidone, 1-propanol, 1,3- and 1,5-propanediol, 2-propanol, propanoic acid, propylene glycol, pyridine, tetrahydrofuran, triethylene glycol, 1,2-dimethylhydrazine, or a mixture thereof.

58. The method of claim 56, wherein the liquid phase further comprises one or more water-soluble, partially water-soluble, or water-dispersible ingredient, which is an acid, a base, a salt, a water-soluble polymer, tetraethoxyorthosilicate (TEOS), or a dendrimer or polymer that make micelles, or a mixture thereof.

59. (canceled)

60. (canceled)

61. The method of claim 49, wherein the emulsion is an oil-in-water emulsion (O/W), a water-in-oil (W/O) emulsion, a bicontinuous emulsion, or a multiple emulsion.

62-122. (canceled)

123. The method of claim 49, wherein the emulsion and the suspension are used in an emulsion volume to cellulose I nanocrystals mass ratio from about 1 to about 30 ml/g to form the mixture of step c).

124. The method of claim 49, wherein the porogen has not sufficiently evaporated during spray-drying to form pores in the microparticles, and wherein step e) is carried out by evaporating the porogen or by leaching the porogen out of the microparticles.

125. (canceled)

126. (canceled)

127. (canceled)

128. (canceled)

129. The method of claim 49, wherein the porogen has sufficiently evaporated during spray-drying to form pores in the microparticles, and wherein step e) is not carried out.
